(19) United States
(12) Patent Application Publication
(10) Pub. No.: US 2014/0271470 A1
(43) Pub. Date: Sep. 18, 2014

(54) TARGETED THERANOSTICS FOR METASTATIC PROSTATE CANCER

(71) Applicants: Laurel O. Sillerud, Albuquerque, NM (US); David L. Vander Jagt, Albuquerque, NM (US); Lorraine Deck, Albuquerque, NM (US)

(72) Inventors: Laurel O. Sillerud, Albuquerque, NM (US); David L. Vander Jagt, Albuquerque, NM (US); Lorraine Deck, Albuquerque, NM (US)

(21) Appl. No.: 14/206,848

(22) Filed: Mar. 12, 2014

Related U.S. Application Data
(60) Provisional application No. 61/778,622, filed on Mar. 13, 2013.
(60) Provisional application No. 61/810,119, filed on Apr. 9, 2013.

(51) Int. Cl. A61K 47/48; A61K 51/04; A61K 49/18; A61K 49/00; A61K 31/337; A61K 45/06

(52) U.S. Cl. CPC A61K 47/48723; A61K 31/337; A61K 45/06; A61K 49/1878; A61K 49/0058; A61K 51/0491
USPC 424/1.73; 424/491; 424/130.1; 424/9.3; 424/9.6

(57) ABSTRACT

The present invention relates to methods of diagnosing and treating prostate cancer, including metastatic prostate cancer. Related pharmaceutical compositions are also provided.

### CROSS REFERENCE TO RELATED APPLICATIONS

This application claims priority from U.S. Provisional Patent Application Ser. No. 61/778,622, filed on Mar. 13, 2013 and entitled “Targeted Theranostics for Metastatic Prostate Cancer” and U.S. Provisional Patent Application Ser. No. 61/810,119, filed on Apr. 9, 2013 and entitled “Quantitative MRI of Superparamagnetic Iron Oxide Nanoparticles (SPIONs) Targeted to Prostate Specific Membrane Antigen in Human Prostate Tumor Xenografts”. The complete contents of both of these provisional applications are hereby incorporated by reference in their entirety.

### STATEMENT REGARDING FEDERAL FUNDING

There is no government support at this time.

### FIELD OF THE INVENTION

The present invention relates to methods of diagnosing and treating prostate cancer, including metastatic prostate cancer. Related pharmaceutical compositions are also provided.

### BACKGROUND OF THE INVENTION

Prostate cancer is a major cause of cancer-related deaths in the United States; the number of patients dying each year is in excess of 30,000 (Nelson et al., 2003). Early diagnosis is a key to successful intervention. Current methods, such as MRI or ultrasound, for imaging and staging prostate cancer lack specificity because they do not incorporate known biological features of the disease into their procedures. For example, MRI often reveals a plethora of suspect nodules in the prostate which turn out later to be benign. We propose to use multiple specific recognition ligands conjugated to magnetic nanoparticles (SIPPs), which will specifically target cell surface epitopes and allow us to image and treat both organ-confined and disseminated disease. The current standard diagnostic method for early prostate cancer detection relies upon a combination of digital rectal examination and serum prostate-specific antigen (PSA) measurement. Serum PSA can rise to levels of concern from a number of situations other than cancer, such as from benign prostatic hyperplasia (BPH). Since BPH becomes more prevalent with advancing age, prostate cancer is often found against a BPH background. Data from the prostate cancer prevention trial (Thompson et al., 2004) has shown that prostate cancer was detected in roughly 25% of the patients who had normal PSA levels and examinations, suggesting that elevated PSA may relate more to benign prostate (BPH) volume than prostate cancer (Stamey 2003; Stamey et al., 2002). It is well-known that, while the sensitivity of PSA testing is ˜90% for the diagnosis of prostate cancer, the specificity is only 36% (Song et al. 2005) for a [PSA] cutoff of 4 ng/ml. Approximately 40% of prostate cancer patients, who elect to undergo a radical prostatectomy, are found to be understaged after histopathological examination of the resected tissue. This is thought to be the reason why there is a 25% rate of recurrence of prostate cancer after radical prostatectomy. Recurrent disease is often metastatic, widely disseminated, and becomes androgen independent and drug resistant, leading to mortality within 12-33 months. The present invention is directed to SIPPs encapsulated with therapeutic regimes and conjugated to ligand recognition molecules specific for cell surface markers (e.g. PSMA) to allow direct delivery of drugs (especially poorly bioavailable chemotherapeutic agents such as paclitaxel, docetaxel, other compounds) to metastatic, drug resistant prostate tumors. By combining an inhibitor of the pro-survival transcription factor NF-κB with a taxane such as paclitaxel, the inventors propose this drug combination as treatment for metastatic tumors that are resistant to taxanes. This is based upon recent demonstration that activation of the pro-survival NF-κB signaling pathway contributes to the development of resistance to taxanes (Sprowl et al., 2012; O'Neill et al., 2011; Caicedo-Granados et al., 2011; Fujiwara et al., 2011; Sreekanth et al., 2011).

### SUMMARY OF THE INVENTION

In one embodiment, the invention provides an immunomicelle comprising:

(a) a particulate core comprising a mixture of superparamagnetic particles and a bioactive agent or drug, including a taxane and a NF-κB drug (i.e., a drug which is modified by incorporating lipids, such as C₄-C₁₈ lipids or fatty acids on the drugs), preferably at least one anti-cancer active agent said core being encapsulated by a plurality of phospholipids comprising at least one pegylated phospholipid (preferably, stealth inducing as otherwise described herein), a phospholipid comprising conjugation functionalities (“a conjugatable phospholipid”, e.g., a biotinylated PEG phospholipid, among other conjugatable phospholipids, preferably pegylated phospholipids), and optionally, a fluorescence-inducing (fluorescent) phospholipid, and/or a cross-linking agent, including a cross-linking phospholipid; and
(b) a targeting antibody or peptide or other binding motif (e.g. an antibody which binds to PSMA) prostate cancer, including metastatic prostate cancer) which is/are conjugated to said particulate core through an appropriate functionality of the conjugatable phospholipid (such that the antibody is preferably disposed at the surface of the immunomicelle).

In one embodiment of the immunomicelle formulation:

(a) the superparamagnetic particles are superparamagnetic iron platinum particles (SIPP), superparamagnetic iron oxide nanoparticles (SPIONs) or superparamagnetic manganese oxide particles (SMIONs);
(b) the optional active ingredient/agent is an anticancer active agent selected from the group consisting of paclitaxel, docetaxal, doxorubicin, oxaliplatin, cisplatin, mitoxantrone and bevacizumab; among others (as described in greater detail herein) and optionally and preferably in combination with an inhibitor of the NF-κB pathway; and
(c) the targeting antibody or peptide is an antibody which binds to PSMA.

In certain embodiments, the immunomicelle particulate core comprises an agent or combination of agents that can be used for both magnetic resonance imaging and computed tomography to diagnose, image, and/or determine the stage of a cancer. The particulate core is preferably a superparamagnetic iron platinum nanoparticle (SIPP) that can be used for dual MRI and CT imaging and diagnosis. Platinum has a high x-ray absorption coefficient of 6.95 cm2/g at 50 KeV, making the particles useful as CT contrast agents and MRI contrast agents.

In certain embodiments, the encapsulated particulate cores described herein each have an average diameter of between about 10 nm and 1000 nm, preferably about 15 to about 150 nm, about 20 to about 100 nm, about 25 to about 75 nm, more preferably between about 30 to about 70 nm, even more preferably between about 40 to about 60 nm, and even more preferably around 50 nm.

In other embodiments, the invention provides a pharmaceutical formulation comprising a plurality of the PEGylated immunomicelles as described herein, wherein the encapsulated particulate cores of each of said immunomicelles are preferably cross-linked, preferably by UV-light initiated polymerization.

In still other embodiments, the immunomicelle particulate core further comprises an agent or combination of agents for the treatment of cancer, especially prostate cancer and even more preferably metastatic prostate cancer.

In still other embodiments, the immunomicelle particulate core further comprises more than one anticancer agent (at least two) or a “cocktail” for treating cancer in a patient or subject.

In still other embodiments of the present invention, the bioactive agents or drugs are modified with lipids to produce “lipid modified drugs”, for example, by conjugating C₄-C₁₈ lipids or fatty acids through, for example, ester or amide groups, among others to provide prodrug forms of the bioactive agents or drugs. In practice, the lipid modified drugs will not release the active drug until the lipid chains are cleaved off of the lipid modified drugs within the lysosome of the cells to be targeted by the drug.

In still other embodiments, the invention provides a method of simultaneously treating and imaging prostate cancer, including metastic prostate cancer, comprising administering to a subject in need thereof a pharmaceutical formulation comprising a plurality of immunomicelles as described herein. In certain embodiments, methods of treatment of the invention are used to treat and image prostate cancer, in particular metastatic prostate cancer.

In still other embodiments, the invention provides a method of simultaneously treating and imaging prostate cancer comprising co-administering to a subject in need thereof a pharmaceutical formulation comprising a plurality of the immunomicelles as described herein and one or more additional anti-cancer active ingredients. In preferred embodiments the anti-cancer agent is a taxane, such as paclitaxel or docetaxel, optionally and preferably in combination with a NF-κB pathway inhibitor as otherwise described herein.

In still other embodiments, the invention provides a method of diagnosing the presence and/or progression in a subject of prostate cancer comprising:

(a) administering a pharmaceutical formulation of the invention to the subject;
(b) subjecting the subject to magnetic resonance imaging; and
(c) determining through MRI contrast enhancement whether the subject suffers from prostate cancer and in particular, metastatic prostate cancer by comparing the resulting MRI image from the subject with a control or standard (which may be a disease control or a normal/healthy control to which the subject's MRI image may be compared for diagnosis). It is noted that a control may be an MRI, a read-out of an MRI or other data which may be readily used to compare the MRI of a subject or patient with either a normal/healthy patient or a patient with disease in varying states, as applicable.

Certain embodiments of these diagnostic methods further comprise measuring in a subject diagnosed with prostate cancer both the MRI contrast enhancement of the tumor and the tumor volume. Other embodiments of the diagnostic methods determine the ability of the formulation to decrease the volume of the tumor and to cause contrast enhancement of the tumor, when compared to a control substance.

In other embodiments, the invention provides a method of determining the existence of cancer tissue in a patient comprising administering to said patient an effective amount of a population of paramagentic nanoparticles and subjecting said nanoparticles to NMR relaxometry to determine the volumetric quantitative MRI measurement of any superparamagnetic nanoparticle in biological tissues. Preferably, MRI measurements are taken of T₁-weighted (T₁) and T₂-weighted (T_{2w}) images, the background relaxation times (T₁, T₂) of the tissue of interest and the relaxivity of the nanoparticles. The T_{1w}, and T_{2w} images are then converted into contrast images; and the contrast images are subtracted to yield the contrast difference.

Thus, the invention in certain embodiments provides novel immunomicelles that are specifically targeted to prostate cancer tissue for both imaging and therapy, and that are also useful in the diagnosis and treatment of prostate cancer. For example, monoclonal antibodies against PSMA, conjugated to crosslinked and PEGylated lipid micelles containing magnetic nanoparticles and the therapeutic agent paclitaxel, will target the therapeutic and diagnostic (theranostic) agents concurrently to prostate cancer tissue, including metastatic prostate cancer tissue, allowing for specific imaging using MRI and targeted therapy.

As described herein, we have synthesized immunomicelles comprising superparamagnetic iron platinum particles (SIPP), superparamagnetic iron oxide nanoparticles (SPIONs) or superparamagnetic manganese oxide particles (SMIONs) for use as therapeutics in the treatment of primary and metastatic prostate cancer and for use as in vivo imaging agents in detecting primary and metastatic prostate cancer.

In one embodiment, the invention provides novel superparamagnetic iron platinum nanoparticles (SIPPs: Taylor et al., 2011; 2012) conjugated to anti-PSMA antibodies that recognize prostate cancer tissue and use these nanoparticles to measure and treat prostate cancer. Conjugated SIPPs of the invention enable the measurement of the tumor during treatment with drugs that target the pro-inflammatory NFκB pathway (such as resveratrol, LD-55 and other related compounds) and directly demonstrate their efficacy in vivo. Since the SIPPs are non-toxic, they are broadly applicable in treating a wide array of human diseases.

In certain embodiments of the invention, nanoparticles of superparamagnetic iron oxide nanoparticles (SPIONS) which may be polydisperse or monodisperse (i.e., particles are all or nearly all the same size) are conjugated to an antibody which binds APP, tau protein or beta amyloid. The SPIONs are preferably magnetite (SiMAG-TCL (Chemicell, Berlin, Germany) which are conjugated with a conjugating agent such as N-hydroxysulfosuccinimide (Sulfo-NHS) and 1-Ethyl-3-[3-dimethylaminopropyl]carbodiimide hydrochloride (EDC), coupled to an antibody which binds to PSMA to diagnose prostate cancer and/or assess the progress of treatment of prostate cancer in a patient, among other methods.

Compositions according to the present invention may be formulated in pharmaceutical dosage form (often as an oral or parenteral dosage form) and delivered to the patient or subject to be diagnosed and/or treated. Diagnosis occurs by magnetic resonance imaging.

In another embodiment, the invention provides a PSMA-targeted nanoplex comprising:

(a) a radiolabel for detection;
(b) a siRNA delivery vector comprising a siRNA which downregulates a specific pathway;
(c) a prodrug-activating enzyme that synthesizes a cytotoxic drug locally from a systemically administered nontoxic drug at a site targeted by the nanoplex; and
(d) a PSMA targeting moiety.

These and other aspects of the invention are described further in the Detailed Description of the Invention.

### BRIEF DESCRIPTION OF THE DRAWINGS

FIG. 1. An electron microscope image (100,000×) of Miltenyi μMACS SPIONs. The light blue scale bar is 20 nm long. As determined in the experiment of Example 1.

FIGS. 2A and B. The relationship between the measured iron concentration and the longitudinal (R₁) and transverse (R₂) water relaxation rates in 1% agarose gels (filled symbols) containing MACS beads (A), and (open symbols) anti-PSMA conjugated μMACS beads bound to LNCaP cells in 1 agarose (B). The error bars reflect the standard errors from the fits to the relaxation time measurements. Note that the relaxivity of beads does not depend on whether or not they are bound to cells. As determined in the experiment of Example 1.

FIG. 3. T1w and T2w NMR images of slice through LNCaP tumor. A. Control T₁-w pre-contrast, B. Control T₂-w contrast, C. T₁-w 20 hours post-contrast, D. T₂-w 20 hours post-contrast. The tumor is circled in a). In c), enhancement is heterogeneous, showing a few obvious regions of bright contrast. In D, substantial areas of dark contrast are visible, indicating that the contrast agent has diffused to regions of the tumor that show insignificant enhancement in C. As determined in the experiment of Example 1.

FIG. 4. Multiple MR T2w image slices after injection of anti-PSMA conjugated SPIONs into a LNCaP human prostate tumor xenograft in a nude mouse. These images were taken 22 hours after the injection. As determined in the experiment of Example 1.

FIG. 5. (A) Contrast as a function of [Fe]. (B) Inversion of the Contrast Difference function. As determined in the experiment of Example 1.

FIG. 6. Quantitative maps of the iron concentration in a LNCaP human prostate tumor xenograft in a nude mouse. Top: Control image taken prior to the injection of SPIONs. Note the large tumor centered near (x,y)=(40,70) in the image. The iron background is less than 5 likely due to blood from the hypoxic regions within the tumor. Bottom: Iron image taken 22 hours after the injection of anti-PSMA conjugated SPIONs into the tumor; iron concentration rose to ˜80 μM at (40, 100). As determined in the experiment of Example 1.

FIG. 7. Quantitative Iron image of LNCaP tumor slice 22 hours after injection of anti-PSMA conjugated SPIONs into a LNCaP human prostate tumor xenograft in a nude mouse. The iron within the tumor appears bright due to the fact that the contrast difference is always positive (See equation X and FIG. 2). As determined in the experiment of Example 1.

FIG. 8. Mathematical plots of slice, time series. As determined in the experiment of Example 1.

FIG. 1A. Growth inhibition (A) and induction of cell death (B) in LNCaP and C4-2 human prostate cancer cells by ca27. Cell growth and death were determined by total cell counts and trypan blue positive cell counts, respectively. Cells were cultured in the presence of ca27 for 96 hours. Bars represent the average of quadruplicate values+standard deviation. Cell growth and cell viablitity are expressed as percent of control (0.1% DMSO). * denote P<0.05 compared to 0.1% DMSO control. As determined in the experiment of Example 2.

FIG. 2A. (A) Rapid down-regulation of AR protein by ca27 in LNCaP cells. (B) Inhibition of PSA mRNA by ca27 in LNCaP cells. Bars represent the average of quadruplicate values+standard deviation. Control=0.1% DMSO; * denote P<0.05 compared to 0.1% DMSO control. As determined in the experiment of Example 2.

FIG. 2A1. Photomicrographs of LNCaP (Left) and DU145 (Right) human prostate tumor xenograft sections grown in nude mice and excised. The blue stain in the LNCaP section arises from antiPSMA-antibody-labeled SPIONs which were injected into the tail vein of the tumor-bearing mouse 24 hours earlier. Note the lack of blue staining in the DU145 tumor section due to the fact that DU145 tumors do not express PSMA (Table 1). As determined in the experiment of Example 2.

FIG. 3A. MR images taken of a nude mouse bearing a human LNCaP xenograft (indicated by arrows) before (Left) and after (Right) the injection into a tail vein of 100 μL of anti-PSMA antibody bearing SPIONs. Note the tumor on the lower right and its brightening in the right-hand image. As determined in the experiment of Example 2.

FIG. 4A. Time course of image intensity changes in a nude mouse with 2 LNCaP tumors injected with antiPSMA SPIONs. Intensity data are shown for the tail vein (cyan), muscle control (yellow), and the averages for two slices for each tumor (Pink/blue). As determined in the experiment of Example 2.

FIG. 5A. (Above) TEM and DLS of SIPP Cores and DSPE SIPPs. TEM images of (a) SIPP cores and (b, c) DSPE-SIPPs. Scale bars are 20 nm, 50 nm, and 50 nm, respectively. Arrows denote internal areas of the DSPE-SIPPs where space can be seen between the hydrophobic SIPP cores. (d) DLS of a 1:50 dilution of DSPE-SIPPs in PBS. As determined in the experiment of Example 2.

FIG. 6A. Confocal images of PSMA-targeted, rhodamine-red-containing super-paramagnetic phospholipid micelles (SPMs) containing fluorescent paclitaxel (green) (Top Row) and control IgG-SPMs (Bottom Row) incubated with C4-2 human prostate cancer cells and stained with DAPI. The last column on the right shows the summed images which contain all three colors for the J591-SPMs, and only DAPI staining for the IgG-SPMs. As determined in the experiment of Example 2.

FIG. 7A. (Right) Biodistribution of SIPPs loaded with paclitaxel in C4-2 tumor-bearing nude mice showing targeting to the tumors via J591. Paclitaxel in the SPMs was assayed by ELISA of the indicated excised tissues. Note that targeting with J591 markedly increased the PTX in the tumors vs. nontargeted IgG SPMs (n=6). As determined in the experiment of Example 2.

FIG. 8A. Tumor volume growth curves for nude mice bearing human C4-2 prostate cancer xenografts treated with various preparations of SIPPs. (A) Black squares, no treatment controls. (B) Red squares, Targeted SIPPs without drug, showing no effect on tumor growth. (C) Blue squares, SIPPs containing paclitaxel targeted with a control IgG antibody showing no effect on tumor growth. (D) Green triangles, paclitaxel alone, without SIPPs showing the efficacy of this chemotherapeutic drug by itself. (E) Purple squares, SIPPs containing paclitaxel, targeted to PSMA, showing that targeting specifically brings the drug to the tumors and prevents their growth. As determined in the experiment of Example 2.

FIG. 1XA. SPECT imaging of SCID mouse bearing Pip (PSMA+ve) and Flu (PSMA−ve) tumor. Mouse was injected i.v. with 1.4 mCi of 111In labeled PSMA-targeted nanoplex (150 mg/kg in 0.2 ml). SPECT images were sec/projection. Following tomography, CT images were acquired in 512 projections to allow coregistration. Volume-rendered images were created using Amira image processing software. Decay-corrected volume-rendered SPECT/CT images at 48 h and 72 h demonstrate high liver uptake and specific accumulation in PSMA expressing Pip tumor. FIG. 1XB. Nanoplex concentration in Pip and Flu tumors without (top panel) and with blocking (bottom panel). For the blocking studies 100 μg of anti-PSMAmouse monoclonal antibody (Clone GCP-05, Abcam) were injected i.v. in a PC3-Pip and PC3-Flu tumor bearing mouse. Five hours after injection, 1.5 mg of nanoplex (75 mg/kg) were injected i.v. in the same mouse. Mice were sacrificed 48 h after nanoplex injection. Tumors, muscle and kidney were excised and imaged on the Xenogen Spectrum system to detect rhodamine present in the nanoplex. Images are scaled differently for unblocked and blocked tissues. As determined in the experiment of Example 3.

FIG. 2X. In vivo tCho maps from 2D CSI data sets acquired from a PC3-Pip tumor (˜400 mm3) before, 24 h, and 48 h after i.v. injection of the PSMA-targeted nanoplex (150 mg/kg) carrying bCD and Chk-siRNA. B. tCho concentration calculated in arbitrary units before, 24 h, and 48 h after injection of nanoplex. Parameters used were echo time (TE)=120 ms, repetition time (TR)=1000 ms, 4 scans per phase encode step. CSI spectra were acquired at 9.4 T with an in-plane spatial resolution of 1 mm×1 mm from a 4 mm-thick slice. As determined in the experiment of Example 3.

FIG. 3X. In vivo 19F MR spectra acquired from a PC3-Pip tumor (˜400 mm3) at (A) 24 h and (B) 48 h after i.v. injection of the PSMA-targeted nanoplex (150 mg/kg) carrying bCD and Chk-siRNA. Spectra were acquired after a combined i.v. and i.p. injection of 5-FC (450 mg/kg), on a Bruker Biospec 9.4 T spectrometer using a 1 cm solenoid coil tunable to 1H and 19F frequency. Following shimming on the water proton signal, serial nonselective 19F MR spectra were acquired with a repetition time of 0.8 s, number of scans, 2,000; spectral width, 10 KHz. As determined in the experiment of Example 3.

### DETAILED DESCRIPTION OF THE INVENTION

The following terms are used throughout the specification to describe the present invention. Where a term is not given a specific definition herein, that term is to be given the same meaning as understood by those of ordinary skill in the art. The definitions given to the disease states or conditions which may be treated using one or more of the compounds according to the present invention are those which are generally known in the art.

It is noted that, as used in this specification and the appended claims, the singular forms “a,” “an,” and “the,” include plural referents unless expressly and unequivocally limited to one referent. Thus, for example, reference to “a compound” includes two or more different compound. As used herein, the term “include” and its grammatical variants are intended to be non-limiting, such that recitation of items in a list is not to the exclusion of other like items that can be substituted or other items that can be added to the listed items.

The term “patient” or “subject” is used throughout the specification to describe an animal, preferably a human, to whom treatment, including prophylactic treatment, with the compositions according to the present invention is provided (a patient or subject in need). For treatment of those infections, conditions or disease states which are specific for a specific animal such as a human patient, the term patient refers to that specific animal. In many instances, diagnostic methods are applied to patients or subjects who are suspected of having cancer or a neuroinflammatory disease or who have cancer or a neuroinflammatory disease and the diagnostic method is used to assess the severity of the disease state or disorder.

The term “compound” is used herein to refer to any specific chemical compound disclosed herein. Within its use in context, the term generally refers to a single small molecule as disclosed herein, but in certain instances may also refer to stereoisomers and/or optical isomers (including racemic mixtures) of disclosed compounds. The term compound includes active metabolites of compounds and/or pharmaceutically active salts thereof.

The term “effective amount” is used throughout the specification to describe concentrations or amounts of formulations or other components which are used in amounts, within the context of their use, to produce an intended effect according to the present invention. The formulations or component may be used to produce a favorable change in a disease or condition treated, whether that change is a remission, a favorable physiological result, a reversal or attenuation of a disease state or condition treated, the prevention or the reduction in the likelihood of a condition or disease-state occurring, depending upon the disease or condition treated. Where formulations are used in combination, each of the formulations is used in an effective amount, wherein an effective amount may include a synergistic amount. The amount of formulation used in the present invention may vary according to the nature of the formulation, the age and weight of the patient and numerous other factors which may influence the bioavailability and pharmacokinetics of the formulation, the amount of formulation which is administered to a patient generally ranges from about 0.001 mg/kg to about 50 mg/kg or more, about 0.5 mg/kg to about 25 mg/kg, about 0.1 to about 15 mg/kg, about 1 mg to about 10 mg/kg per day and otherwise described herein. The person of ordinary skill may easily recognize variations in dosage schedules or amounts to be made during the course of therapy.

The term “active ingredient” as used herein is defined to include a pharmaceutically acceptable salt, enantiomer, stereoisomer, solvate or polymorph of any active ingredient described herein.

The term “prophylactic” is used to describe the use of a formulation described herein which reduces the likelihood of an occurrence of a condition or disease state in a patient or subject. The term “reducing the likelihood” refers to the fact that in a given population of patients, the present invention may be used to reduce the likelihood of an occurrence, recurrence or metastasis of disease in one or more patients within that population of all patients, rather than prevent, in all patients, the occurrence, recurrence or metastasis of a disease state.

The term “pharmaceutically acceptable” refers to a salt form or other derivative (such as an active metabolite or prodrug form) of the present compounds or a carrier, additive or excipient which is not unacceptably toxic to the subject to which it is administered.

The term “prostate cancer” is used to describe a disease in which cancer develops in the prostate, a gland in the male reproductive system. It occurs when cells of the prostate mutate and begin to multiply uncontrollably. These cells may metastasize (metastatic prostate cancer) from the prostate to virtually any other part of the body, particularly the bones and lymph nodes, but the kidney, bladder and even the brain, among other tissues. Prostate cancer may cause pain, difficulty in urinating, problems during sexual intercourse, erectile dysfunction. Other symptoms can potentially develop during later stages of the disease.

Treatment options for prostate cancer with intent to cure are primarily surgery and radiation therapy. Other treatments such as hormonal therapy, chemotherapy, proton therapy, cryosurgery, high intensity focused ultrasound (HIFU) also exist depending on the clinical scenario and desired outcome. The present invention may be used to enhance any one or more of these therapies or to supplant them.

An important part of evaluating prostate cancer is determining the stage, or how far the cancer has spread. Knowing the stage helps define prognosis and is useful when selecting therapies. The most common system is the four-stage TNM system (abbreviated from Tumor/Nodes/Metastases). Its components include the size of the tumor, the number of involved lymph nodes, and the presence of any other metastases.

The most important distinction made by any staging system is whether or not the cancer is still confined to the prostate or is metastatic. In the TNM system, clinical T1 and T2 cancers are found only in the prostate, while T3 and T4 cancers have spread elsewhere and metastasized into other tissue. Several tests can be used to look for evidence of spread. These include computed tomography to evaluate spread within the pelvis, bone scans to look for spread to the bones, and endorectal coil magnetic resonance imaging to closely evaluate the prostatic capsule and the seminal vesicles. Bone scans often reveal osteoblastic appearance due to increased bone density in the areas of bone metastasis—opposite to what is found in many other cancers that metastasize. Computed tomography (CT) and magnetic resonance imaging (MRI) currently do not add any significant information in the assessment of possible lymph node metastases in patients with prostate cancer according to a meta-analysis.

Prostate cancer is relatively easy to treat if found early. After a prostate biopsy, a pathologist looks at the samples under a microscope. If cancer is present, the pathologist reports the grade of the tumor. The grade tells how much the tumor tissue differs from normal prostate tissue and suggests how fast the tumor is likely to grow. The Gleason system is used to grade prostate tumors from 2 to 10, where a Gleason score of 10 indicates the most abnormalities. The pathologist assigns a number from 1 to 5 for the most common pattern observed under the microscope, and then does the same for the second most common pattern. The sum of these two numbers is the Gleason score. The Whitmore-Jewett stage is another method sometimes used. Proper grading of the tumor is critical, since the grade of the tumor is one of the major factors used to determine the treatment recommendation.

Advanced prostate cancer can spread to other parts of the body and this may cause additional symptoms. The most common symptom is bone pain, often in the vertebrae (bones of the spine), pelvis or ribs. Spread of cancer into other bones such as the femur is usually to the proximal part of the bone. Prostate cancer in the spine can also compress the spinal cord, causing leg weakness and urinary and fecal incontinence.

Prostate cancer is classified as an adenocarcinoma, or glandular cancer, that begins when normal semen-secreting prostate gland cells mutate into cancer cells. The region of prostate gland where the adenocarcinoma is most common is the peripheral zone. Initially, small clumps of cancer cells remain confined to otherwise normal prostate glands, a condition known as carcinoma in situ or prostatic intraepithelial neoplasia (PIN). Although there is no proof that PIN is a cancer precursor, it is closely associated with cancer. Over time these cancer cells begin to multiply and spread to the surrounding prostate tissue (the stroma) forming a tumor. Eventually, the tumor may grow large enough to invade nearby organs such as the seminal vesicles or the rectum, or the tumor cells may develop the ability to travel in the bloodstream and lymphatic system. Prostate cancer is considered a malignant tumor because it is a mass of cells which can invade other parts of the body. This invasion of other organs is called metastasis. Prostate cancer most commonly metastasizes to the bones, lymph nodes, rectum, and bladder.

In addition to therapy using the compounds according to the present invention, therapy (including prophylactic therapy) for prostate cancer supports roles in reducing prostate cancer for dietary selenium, vitamin E, lycopene, soy foods, vitamin D, green tea, omega-3 fatty acids and phytoestrogens. The selective estrogen receptor modulator drug toremifene has shown promise in early trials. Two medications which block the conversion of testosterone to dihydrotestosterone (and reduce the tendency toward cell growth), finasteride and dutasteride, are shown to be useful. The phytochemicals indole-3-carbinol and diindolylmethane, found in cruciferous vegetables (califlower and broccholi), have favorable antiandrogenic and immune modulating properties. Prostate cancer risk is decreased in a vegetarian diet.

Treatment for prostate cancer may involve active surveillance, surgery (prostatecomy or orchiectomy), radiation therapy including brachytherapy (prostate brachytherapy) and external beam radiation as well as hormonal therapy. There are several forms of hormonal therapy which include the following, each of which may be combined with compounds according to the present invention.
Antiandrogens such as flutamide, bicalutamide, nilutamide, and cyproterone acetate which directly block the actions of testosterone and DHT within prostate cancer cells.
Medications such as ketoconazole and aminoglutethimide which block the production of adrenal androgens such as DHEA. These medications are generally used only in combination with other methods that can block the 95% of androgens made by the testicles. These combined methods are called total androgen blockade (TAB), which can also be achieved using antiandrogens.
GnRH modulators, including agonists and antagonists. GnRH antagonists suppress the production of LH directly, while GnRH agonists suppress LH through the process of downregulation after an initial stimulation effect. Abarelix is an example of a GnRH antagonist, while the GnRH agonists include leuprolide, goserelin, triptorelin, and buserelin.
The use of abiraterone acetate can be used to reduce PSA levels and tumor sizes in aggressive end-stage prostate cancer for as high as 70% of patients. Sorafenib may also be used to treat metastatic prostate cancer.

Each treatment described above has disadvantages which limit its use in certain circumstances. GnRH agonists eventually cause the same side effects as orchiectomy but may cause worse symptoms at the beginning of treatment. When GnRH agonists are first used, testosterone surges can lead to increased bone pain from metastatic cancer, so antiandrogens or abarelix are often added to blunt these side effects. Estrogens are not commonly used because they increase the risk for cardiovascular disease and blood clots. The antiandrogens do not generally cause impotence and usually cause less loss of bone and muscle mass. Ketoconazole can cause liver damage with prolonged use, and aminoglutethimide can cause skin rashes.

Bone pain due to metastatic disease is treated with opioid pain relievers such as morphine and oxycodone. External beam radiation therapy directed at bone metastases may provide pain relief Injections of certain radioisotopes, such as strontium-89, phosphorus-32, or samarium-153, also target bone metastases and may help relieve pain.

Additional prostate drugs which can be used in combination with the compositions according to the present invention and include, for example, the enlarged prostate drugs/agents, as well as eulexin, flutamide, goserelin, leuprolide, lupron, nilandron, nilutamide, zoladex and mixtures thereof. Enlarged prostate drugs/agents as above, include for example, ambenyl, ambophen, amgenal, atrosept, bromanyl, bromodiphenhydramine-codeine, bromotuss-codeine, cardura, chlorpheniramine-hydrocodone, ciclopirox, clotrimazole-betamethasone, dolsed, dutasteride, finasteride, flomax, gecil, hexalol, lamisil, lanased, loprox, lotrisone, methenamine, methen-bella-meth Bl-phen sal, meth-hyos-atrp-M blue-BA-phsal, MHP-A, mybanil, prosed/DS, Ro-Sed, S-T Forte, tamsulosin, terbinafine, trac, tussionex, ty-methate, uramine, uratin, uretron, uridon, uro-ves, urstat, usept and mixtures thereof.

The term “neoplasia” refers to the uncontrolled and progressive multiplication of tumor cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive and which express PSMA. Thus, neoplasia includes “cancer”, which herein refers to a proliferation of tumor cells having the unique trait of loss of normal controls, resulting in unregulated growth, lack of differentiation, local tissue invasion, and/or metastasis.

As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, cancers which express PSMA, including carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991. In the present invention, the methods are principally directed to diagnosing and monitoring therapy in PSMA expressing cancers, preferably prostate cancer and/or metastatic prostate cancer, but numerous other cancer tissue may be identified, diagnosed or treatment monitored by the method(s) of the present invention.

Formulations of the invention may include a pharmaceutically acceptable diluent, carrier, solubilizer, emulsifier, preservative and/or adjuvant. Acceptable formulation materials preferably are nontoxic to recipients at the dosages and concentrations employed. The pharmaceutical formulations may contain materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, polyethylene glycol (PEG), sorbitan esters, polysorbates such as polysorbate 20 and polysorbate 80, Triton, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, REMINGTON'S PHARMACEUTICAL SCIENCES, 18.sup.th Edition, (A. R. Gennaro, ed.), 1990, Mack Publishing Company.

Optimal pharmaceutical formulations can be determined by one skilled in the art depending upon, for example, the intended route of administration, delivery format and desired dosage. See, for example, REMINGTON'S PHARMACEUTICAL SCIENCES, Id. Such formulations may influence the physical state, stability, rate of in vivo release and rate of in vivo clearance of the antibodies of the invention.

Primary vehicles or carriers in a pharmaceutical formulation can include, but are not limited to, water for injection, physiological saline solution or artificial cerebrospinal fluid, possibly supplemented with other materials common in compositions for parenteral administration. Neutral buffered saline or saline mixed with serum albumin are further exemplary vehicles. Pharmaceutical formulations can comprise Tris buffer of about pH 7.0-8.5, or acetate buffer of about pH 4.0-5.5, which may further include sorbitol or a suitable substitute. Pharmaceutical formulations of the invention may be prepared for storage by mixing the selected composition having the desired degree of purity with optional formulation agents (REMINGTON'S PHARMACEUTICAL SCIENCES, Id.) in the form of a lyophilized cake or an aqueous solution. Further, the formulations may be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 5 to about 8.

The pharmaceutical formulations of the invention can be delivered parenterally. When parenteral administration is contemplated, the therapeutic formulations for use in this invention may be in the form of a pyrogen-free, parenterally acceptable aqueous solution. Preparation involves the formulation of the desired immunomicelle, which may provide controlled or sustained release of the product which may then be delivered via a depot injection. Formulation with hyaluronic acid has the effect of promoting sustained duration in the circulation.

Formulations may be formulated for inhalation. In these embodiments, a stealth immunomicelle formulation is formulated as a dry powder for inhalation, or inhalation solutions may also be formulated with a propellant for aerosol delivery, such as by nebulization. Pulmonary administration is further described in PCT Application No. PCT/US94/001875, which describes pulmonary delivery of chemically modified proteins and is incorporated by reference.

Formulations of the invention can be delivered through the digestive tract, such as orally. The preparation of such pharmaceutically acceptable compositions is within the skill of the art. Formulations disclosed herein that are administered in this fashion may be formulated with or without those carriers customarily used in the compounding of solid dosage forms such as tablets and capsules. A capsule may be designed to release the active portion of the formulation at the point in the gastrointestinal tract when bioavailability is maximized and pre-systemic degradation is minimized Additional agents can be included to facilitate absorption. Diluents, flavorings, low melting point waxes, vegetable oils, lubricants, suspending agents, tablet disintegrating agents, and binders may also be employed.

A formulation may involve an effective quantity of a micropoarticle containing formulation as disclosed herein in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

The pharmaceutical composition to be used for in vivo administration typically is sterile. In certain embodiments, this may be accomplished by filtration through sterile filtration membranes. In certain embodiments, where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

Once the formulation of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

Administration routes for formulations of the invention include orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. The pharmaceutical formulations may be administered by bolus injection or continuously by infusion, or by implantation device. The pharmaceutical formulations also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous administration. Compositions according to the present invention, especially those which comprise microparticles containing DNA vectors for gene therapy are preferably administered by intrathecal administration as otherwise in Appendix A hereof.

Molecular imaging seeks to produce quantitative maps of the distribution of particular substances in biological tissues. For this to be accomplished, a quantitative relationship must exist between image intensity or contrast and the concentration of the selected molecular imaging agent. Magnetic resonance imaging (MRI) agents produce image contrast via perturbations of the magnetic relaxation times of nearby nuclei. MRI contrast, however, is a complex, non-linear function of effect of the introduced contrast agent on the transverse and longitudinal nuclear relaxation rates. However, it is possible to measure these nonlinear effects and then to use this empirical calibration to infer the concentration of a given contrast agent in a particular imaging slice.

The field of nanotechnology has progressed to the point where it is possible to target nanoparticles to various sites of biological interest and to deliver cargoes to these sites. One application is the targeted delivery of chemotherapeutic drugs to tumors. We have, for example, loaded superparamagnetic FePt nanoparticles with paclitaxel and shown that they undergo enhanced uptake by prostate tumor xenografts in nude mice (Taylor et al., 2011). If one can use MRI to measure the concentration of nanoparticles, and if one knows the cell surface receptor density for the particle-targets, then one can determine the number of tumor cells in a measured volume. Current MRI in humans enjoys submillimeter resolution so that tumor burden could be measured with good accuracy. The transport of nanoparticles over time could also be shown. We have developed a method for the quantitative MRI of Superparamagnetic Iron Oxide Nanoparticles (SPIONs). This method is applied to an analysis of the time-dependent distribution of anti-Prostate Specific Membrane Antigen (anti-PSMA) conjugated SPIONs within human LNCaP xenografts implanted into the flanks of nude mice. Maps of the distribution of Fe in the tumor show that the SPIONs migrated 3 mm from the injection site in 22 hours. The peak Fe concentration reached within the tumor was 78 μM.

We also describe herein (e.g. in Example 1) a straightforward, NMR relaxometry approach to the volumetric quantitative MRI measurement of any superparamagnetic nanoparticle in biological tissues. The method requires only a simple MRI measurement of T₁-weighted (T_{1w}) and T₂-weighted (T_{2w}) images, the background relaxation times (T₁, T₂) of the tissue of interest and the relaxivity of the nanoparticles. The T_{1w}, and T_{2w} images are then converted into contrast images, which are subtracted to yield the contrast difference. Calibration measurements of the effect of the selected superparamagnetic nanoparticles on water relaxation are used to determine the quantitative relationship between contrast difference and the concentration of nanoparticles. This relationship can be empirically inverted to yield the functional dependence of particle concentration on contrast difference, which is then used to convert the contrast difference image into an absolute nanoparticle concentration image. The method was demonstrated by generating superparamagnetic iron oxide nanoparticles (SPIONs) bearing antibodies directed against human prostate specific membrane antigen. These SPIONs were injected into a PSMA-expressing human LNCaP xenograft in a nude mouse. A series of paired, of T_{1w} and T_{2w} images were then taken and compared with pre-injection control images as indicated above. The image processing pipeline was applied to the data to produce quantitative maps of SPION concentration in each tumor image slice, revealing the time-dependent diffusion and transport of the SPIONs within the tumor.

As described in United States Patent Application Document No. 20110263833 (“Compositions for Isolating a Target Analyte from a Heterogeneous Sample”), the complete contents of which are hereby incorporated by reference, magnetic particles can be permanently magnetizable, or ferromagnetic, or they may demonstrate bulk magnetic behavior only when subjected to a magnetic field. Magnetic particles that exhibit bulk magnetic behavior only when subjected to a magnetic field are “magnetically responsive particles” or are also characterized as “superparamagnetic”. Materials exhibiting bulk ferromagnetic properties, e.g., magnetic iron oxide, may be characterized as superparamagnetic when provided in crystals of about 30 nm or less in diameter.

The superparamagnetic particles used in the instant invention include all of the superparamagnetic particles and superparamagnetic nanoparticles and mixtures thereof described in United States Patent Application Document No. 20110263833. Preferably, a “superparamagnetic particle” is a superparamagnetic iron platinum particle (SIPP) or a superparamagnetic iron oxide nanoparticle (SPION). The term “SPION” refers to a superparamagnetic iron oxide nanoparticle (SPION). Pursuant to the present invention, SPIONS may be polydisperse or monodisperse (i.e., particles are all or nearly all the same size) which are conjugated to an antibody which binds PSMA in order to have SPIONS which are administered to a patient bind and concentrate in cancer tissue which expresses PSMA, especially prostate cancer tissue or metastatic prostate cancer tissue. The SPIONS so concentrated may be used in the MRI methods according to the present invention to evidence the size and extent of cancer tissue, as well as the effect of therapy on the cancer tissue. Thus, methods according to the present invention may be used to diagnose the existence and the extent (including the size) of prostate cancer tissue as well as monitoring the treatment of prostate and other cancer. In certain embodiments, SPIONs are conjugated with a conjugating agent such as N-hydroxysulfosuccinimide (Sulfo-NHS) and 1-Ethyl-3[dimethylaminopropyl]carbodiimide hydrochloride (EDC) and coupled to an anti-PSMA polyclonal or monoclonal antibody which are then used in combination with magnetic resonance imaging to assess in a subject levels of cancer tissue, especially prostate cancer tissue and/or metastatic prostate cancer tissue. Both diagnosis and monitoring of therapy occurs by magnetic resonance imaging as otherwise discussed herein.

In certain embodiments, SPIONS comprise paramagnetic nanoparticles, generally approximately 1-3 nanometers (nm) to about 100 nm, about 5 nm to about 100 nm, about 9-10 nm to about 50 nm, about 5 nm to about 25 nm in diameter which comprise a paramagnetic iron material, preferably ferric oxide (Fe₂O₃), ferrous oxide (FeO) or ferroferric oxide (Fe₃O₄) which is coated with a polymeric coating which is preferably hydrophilic. Preferably, the SPIONS (comprising the iron oxide spheres as well as the polymeric coating) are 1-, 20, 30, 40, 50, 100 or 200 nm in hydrodynamic diameter. The polymeric material which coats the particles may be a hydrophilic polymer such as chitosan, dextran (or any one or more of its pharmaceutically acceptable derivatives such as dextran sulfate and carboxymethyl dextran, among others), starch (or any one or more of its pharmaceutically acceptable derivates such as hydroxyethyl starch, hydroxypropyl starch, cationic starch, hydroxymethylstarch and carboxymethylstarch, among others) or a lipid or phospholipid (e.g., phosphatidylcholine) to protect against aggregation or clumping of the nanoparticles. The hydrodynamic diameter consists of the thickness of the core iron oxide particle as well as the external polymer coating.

In one embodiment, the invention provides a PEGylated stealth immunomicelle comprising:

(a) a particulate core comprising a mixture of superparamagnetic particles and at least one bioactive agent or drug comprising a lipid-modified drug selected from the group consisting of anti-cancer active agents and active agents useful in the treatment of prostate cancer, said core being encapsulated by a plurality of phospholipds comprising at least one pegylated phospholipid, a phospholipid comprising conjugation functionalities, and optionally, a fluorescence-inducing (fluorescent) phospholipid, and/or a cross-linking agent, including a cross-linking phospholipid; and
(b) a targeting antibody or peptide or other binding motif which is selected from the group consisting of a prostate cancer targeting monoclonal or polyclonal antibody and a monoclonal or polyclonal antibody or a peptide which targets prostate specific membrane antigen (PSMA), prostate stem cell antigen (PSCA), the integrin αᵥβ₃, and the neurotensin receptor (NTR) and which is/are conjugated to said particulate core through an appropriate functionality of the conjugatable phospholipid.

The superparamagnetic particles described in the embodiment of the preceding paragraph can be superparamagnetic iron platinum particles (SIPP), superparamagnetic iron oxide nanoparticles (SPIONs) or superparamagnetic manganese oxide particles (SMIONs).

The stealth-inducing PEG phospholipid can be selected from the group consisting of 1,2-distearoyl-sn-glycero-3-phosphoethanolamine-N-[methoxy(polyethylene glycol)-2000] ((DSPE-PEG) or poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl insitol (PI), monosialogangolioside, spingomyelin (SPM), distearoylphosphatidylcholine (DSPC), dimyristoylphosphatidylcholine (DMPC), and dimyristoylphosphatidylglycerol (DMPG), all of which are pegylated;

The conjugated phospholipid can be selected from the group consisting of 1,2-distearoyl-sn-glycero-3-phosphoethanolamine-N-[biotinyl(polyethylene glycol)-2000] ((DSPE-PEG-biotin) 1,2-distearoyl-sn-glycero-3-conjugated phosphoethanolamine-N-[methoxy(polyethylene glycol)-2000] ((DSPE-PEG), conjugated poly(ethylene glycol)-derivatized ceramides (PEG-CER), conjugated hydrogenated soy phosphatidylcholine (HSPC), conjugated egg phosphatidylcholine (EPC), conjugated phosphatidyl ethanolamine (PE), conjugated phosphatidyl glycerol (PG), conjugated phosphatidyl insitol (PI), conjugated monosialogangolioside, conjugated spingomyelin (SPM), conjugated distearoylphosphatidylcholine (DSPC), conjugated dimyristoylphosphatidylcholine (DMPC), and conjugated dimyristoylphosphatidylglycerol (DMPG).

The fluorescence-inducing phospholipid can be a phospholipid comprising a fluorescent moiety, wherein the fluorescent moiety is selected from the group consisting of fluoresceins, rhodamines and rhodols, cyanines, phtalocyanines, squairanines, bodipy dyes, pyrene, anthracene, naphthalene, acridine, stilbene, indole, benzindole, oxazole. benzoxazole, thiazole, benzothiazole, carbocyanine, carbostyryl, prophyrin, salicylate, anthranilate, azulene, perylene, pyridine, quinoline, borapolyazaindacene, xanthene, oxazine, benzoxazine, carbazine, phenalenone, coumarin, benzofuran, benzphenalenone, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA), 4,7-dichlorotetramethylrhodamine (dTAMRA), 4,7-dichlorofluoresceins, 5-carboxyfluorescein (5-FAM) and 6-carboxyfluorescein (6-FAM); and (d) the cross-linking phospholipid is selected from the group consisting of 2-bis(10,12-tricosadiynoyl)-sn-glycero-3-phosphoethanolamine ((Diyne-PE), 1,2-Dioleoyl-sn-Giycero-3-Phosphocholine (DOPC), 1,2-Dilauroyl-sn-Glycero-3-Phosphocholine (DLPC) and 1-Palmitoyl-2-10,12 Tricosadiynoyl-sn-Glycero-3-Phosphocholine (16:0-23:2 DIYNE PC).

In certain embodiments, the encapsulated particulate core has an average diameter of (a) between about 10 to about 1000 nm; or (b) about 15 nm to about 150 nm; or (c) about 25 to about 75 nm.

Pharmaceutical formulations of the invention can comprise a plurality of the PEGylated stealth immunomicelles in combination with a pharmaceutically acceptable carrier, additive or excipient. In certain embodiments, the encapsulated particulate cores of each of the immunomicelles are cross-linked, e.g. by UV-light initiated polymerization.

The invention also provides a method of simultaneously treating and imaging a metastatic prolate cancer tumor comprising administering to a subject in need thereof a pharmaceutical formulation as described in the preceding paragraph. A method of simultaneously treating and imaging a metastatic prolate cancer tumor comprising administering to a subject in need thereof a pharmaceutical formulation as described above is also within the scope of the invention.

In one embodiment, the invention provides a method of diagnosing the presence or progression in a subject of prostate cancer tumor comprising:

(a) administering a formulation comprised of stealth immunomicelles (e.g. PEGylated stealth immunomicelles) to the subject;
(b) subjecting the subject to magnetic resonance imaging; and
(c) determining through MRI contrast enhancement whether the subject suffers from a prostate cancer tumor.

In another embodiment, the method described in the preceding paragraph further comprises measuring in a subject diagnosed with a prostate cancer tumor both the MRI contrast enhancement of the tumor and the tumor volume. Optionally, this method includes the step of determining the ability of the formulation to decrease the volume of the tumor and to cause contrast enhancement of the tumor, when compared to a control substance. The method can further comprise measuring in a subject diagnosed with a prostate cancer tumor both the MRI contrast enhancement of the tumor and the tumor volume. The ability of the formulation to decrease the volume of the tumor and to cause contrast enhancement of the tumor, when compared to a control substance, can also be determined.

The following examples are provided to more fully define and describe the present invention. The examples are not to be taken to limit the scope of the invention of the present application in any way.

### Example 1

### Quantitative MRI of Superparamagnetic Iron Oxide Nanoparticles (SPIONs) Targeted to Prostate Specific Membrane Antigen in Human Prostate Tumor Xenografts

### Methods

SPION conjugation

Sources of chemicals, antibodies

Animals

Cell culture, PSMA+measurement

Xenograft generation

Two tumors in animal, one injected, and the other not

MRI at 1.0 T, anesthesia

Relaxivity measurements

Theory, computed with IDL, Spyglass Transform 3, and Mathematica 6. One simply needs to measure the relaxivity of the SPIONs, to obtain both r1, and r2. A pair of control images are then taken in order to measure the T1 and T2 and signal background of the region of interest. The SPIONs are injected and another pair of images are taken with T1 and T2 weighting. Each of these images are converted into contrast maps using the T1 or T2 backgrounds. The difference between these two contrast maps is an image whose intensity is proportional to the SPION concentration.

$$Signal 1 ( c ) = {e}^{- ( \frac{1}{T 2} + r 2 c ) TE 1} ( 1 - {e}^{- ( \frac{1}{T 1} + r 1 c ) TR 1} )$$

$$BackGround 1 := {e}^{- TE 1 / T 2} ( 1 - {e}^{- TR 1 / T 1} )$$

$$Contrast 1 ( c ) = \frac{( Signal 1 ( c ) - BackGround 1 )}{BackGround 1}$$

$$Signal 2 ( c ) = {e}^{- ( \frac{1}{T 2} + r 2 c ) TE 2} ( 1 - {e}^{- ( \frac{1}{T 1} + r 1 c ) TR 2} )$$

$$BackGround 2 = {e}^{- TE 2 / T 2} ( 1 - {e}^{- TR 2 / T 1} )$$

$$Contrast 2 ( c ) = \frac{( Signal 2 ( c ) - BackGround 2 )}{BackGround 2}$$

$$DeltaContrast = Contrast 1 ( c ) - Contrast 2 ( c )$$

$${S}_{1} ( c ) = {e}^{- ( \frac{1}{{T}_{2}} + {r}_{2} c ) {TE}_{1}} ( 1 - {e}^{- ( \frac{1}{{T}_{1}} + {r}_{1} c ) {TR}_{1}} )$$

$${B}_{1} = {e}^{- {TE}_{1} / {T}_{2}} ( 1 - {e}^{- {TR}_{1} / {T}_{1}} )$$

$${C}_{1} ( c ) = ( {S}_{1} ( c ) - {B}_{1} ) / {B}_{1}$$

$${S}_{2} ( c ) = {e}^{- ( \frac{1}{{T}_{2}} + {r}_{2} c ) {TE}_{2}} ( 1 - {e}^{- ( \frac{1}{{T}_{1}} + {r}_{1} c ) {TR}_{2}} )$$

$${B}_{2} = {e}^{- {TE}_{2} / {T}_{2}} ( 1 - {e}^{- {TR}_{2} / {T}_{1}} )$$

$${C}_{2} ( c ) = ( {S}_{2} ( c ) - {B}_{2} ) / {B}_{2}$$

$$Δ C ( c ) = {C}_{1} ( c ) - {C}_{2} ( c )$$

The difference image, Δc(c), is calibrated by inverting the relationship between contrast and [SPION]. This results in an image whose pixel intensity directly gives the [SPION] within the pixel.

### Results

A. Begin with the theory of spion relaxivity, show how relaxation rates depend on [Fe].
B. Predict contrast vs. [Fe] and MRI timing parameters, t1, t2.
C. Show calibration data taken at 4.7 T. Measure R1, R2 for micromacs.
D. Test theory in a real tumor;

1. Show PSMA expression data and histology for these two cell lines. See that Fe only appears in the PSMA positive xenografts, and not in the DU145s.

2. Do Intratumoral injection of anti-PSMA conjugated SPIONs.

3. Expect T1w bright and T2w dark.

4. This is what we see after injection of SPIONs into tumor.

5. Confirms theory so.

6. Figures here include multiple slices of pre-, and post-SPION injection, processed for [Fe] images. [pick file names for processing: 5553_{—}3,5554_{—}3 (control); 5587_{—}2,5588_{—}4 (post SPION injection)].

E. Then use it in vivo by injecting SPIONs into the circulation of animals containing LNCaP and DU145 human prostate tumor xenografts.

1. SPIONs will target the tumor.

2. SPIONs brighten the LNCaP tumor.

3. Compare results with the control DU145 tumor which shows no brightening.

4. This is a static experiment with only 1 time point.

5. Figures are [Fe] for LNCaP and DU145 tumors showing no increase in MRI signal in the control, DU145 tumors, but a large increase in the LNCaP tumors. [pick file names for processing:

F. Go to a dynamic study over 26 hours.

1. Show time course of brightening.

2. Figures include multi-slice, multi-time [Fe] images. [pick file names for processing:

3. Compare with DU145 time course.

FIG. 1 shows an electron microscope image (100,000×) of Miltenyi μMACS SPIONs which was generated in accordance with the experimental protocol described in this example. The light blue scale bar is 20 nm long.

We also determined the relationship between the measured iron concentration and the longitudinal (R₁) and transverse (R₂) water relaxation rates in 1 agarose gels (filled symbols) containing MACS beads, and (open symbols) anti-PSMA conjugated MACS beads bound to LNCaP cells in 1 agarose, as shown in FIG. 2. The error bars reflect the standard errors from the fits to the relaxation time measurements.

T1w and T2w NMR images of slice through LNCaP tumor were generated, as illustrated in FIG. 3. A. Control T₁-w pre-contrast, B. Control T₂-w contrast, C. T₁-w 20 hours post-contrast, D. T₂-w 20 hours post-contrast. The tumor is circled in a). In c), enhancement is heterogeneous, showing a few obvious regions of bright contrast. In D, substantial areas of dark contrast are visible, indicating that the contrast agent has diffused to regions of the tumor that show insignificant enhancement in C.

Additionally, as shown in FIG. 4, we generated multiple MR T2w image slices after injection of anti-PSMA conjugated SPIONs into a LNCaP human prostate tumor xenograft in a nude mouse. These images were taken 22 hours after the injection. FIG. 5(A) shows contrast as a function of [Fe], and FIG. 5(B) shows the inversion of the contrast difference function.

Quantitative maps of the iron concentration in a LNCaP human prostate tumor xenograft in a nude mouse were produced, as shown in FIG. 6. Top: Control image taken prior to the injection of SPIONs. Note the large tumor centered near (x,y)=(40,70) in the image. The iron background is less than 5 μM, likely due to blood from the hypoxic regions within the tumor. Bottom: Iron image taken 22 hours after the injection of anti-PSMA conjugated SPIONs into the tumor. Here, the iron concentration rose to ˜80 μM at (40, 100). FIG. 7 illustrates a quantitative iron image of LNCaP tumor slice 22 hours after injection of anti-PSMA conjugated SPIONs into a LNCaP human prostate tumor xenograft in a nude mouse. The iron within the tumor appears bright due to the fact that the contrast difference is always positive (see equation X and FIG. 2).

Finally, as shown in FIG. 8, multiple MR Fe image slices after injection of anti-PSMA conjugated SPIONs into a LNCaP human prostate tumor xenograft in a nude mouse. These images were taken 22 hours after the injection. (A) Slice 2; (B) Slice 3; (C) Slice 4; (D) Slice 5; Slice 6.

### Summary

A. We have developed a theory for the dependence of MRI contrast on [Fe].

B. We have calibrated this theory against known samples where we measured T1 and T2 in a control gel, and compared the contrast with that from a series of ICP-confirmed [Fe] samples.

C. We measured the PSMA epitope density on both LNCaP and DU145 human prostate cancer cells and xenografts in nude mice by flow cytometry and RT-PCR.

Developed an epitope-specific MRI contrast agent using SPIONs that targets the contrast to PSMA-expressing human prostate cancer xenografts in nude mice.

D. These results were confirmed via histology of xenografts. See PSMA expression in LNCaP vs. DU145 tissues.

E. We then sought to test the theory against experimental data by injecting SPIONs directly into an LNCaP tumor and following the time course of MRI signal intensity. The results followed our predictions. See Fe in the histology? Of the LNCaP cells?

F. In vivo tail vein injection may be used. Observe brightening of tumor after some hours. No brightening of the DU145 controls.

G. Observe time course of the rise and fall of [Fe] in the tumor.

H. A good contrast agent for PCa detection via MRI is provided. The experiment of this example shows quantitative molecular imaging of SPION iron with an anti-PSMA expressing epitope-specific MRI contrast agent targeted to human prostate cancer xenografts in nude mice

### Example 2

### Targeted Theranostics for Metastatic Prostate Cancer

Treated prostate cancer evolves from an initial androgen-dependent state to one of androgen-independence, with frequent metastases to distant sites, and the development of drug resistance. Although sophisticated magnetic resonance (MR) imaging and spectroscopy methods can aid tumor detection for organ-confined disease (Kurhanewicz et al. 2002; Aydn et al. 2012) the vast majority (>90%) of prostate cancer mortality involves disseminated, metastatic disease. No method currently exists which is specific for targeted detection, imaging, staging and treatment of organ-confined, extracapsular or metastatic prostate cancer. Previous developments in nanoparticle research (Winter et al., 2003a; 2003b; Reimer 2004; Ozawa et al., 2000; Morawski et al., 2004; Johansson et al., 2001; Artemov et al., 2003a; 2003b) supported the possibility of producing multiple superparamagnetic imaging agents for prostate cancer.

We have carried this research forward (Sillerud et al., 2006; Serda et al., 2007; Serda et al. 2008; Taylor et al., 2011; 2012a; 2012b) to demonstrate the successful production of multifunctional superparamagnetic iron platinum nanoparticles (SIPPs) which both recognize prostate tumors, via antibodies directed against prostate specific membrane antigen (PSMA), and eradicate them with incorporated chemotherapeutic agents (paclitaxel; PTX). We have also shown that the incorporation of PTX eradicates these tumors in a specific manner predicated on the presence of PSMA on the tumor cell surfaces.

Our goal was to develop magnetic nanoparticles that targeted prostate tumors for non-invasive detection using a combination of magnetic resonance (MR) and Superconducting Quantum Interference Device (SQUID) imaging. Our hypothesis was that binary mixtures of magnetic imaging agents incorporating two or more prostate cancer cell surface markers, such as prostate specific membrane antigen (PSMA), prostate stem cell antigen (PSCA), the integrin αᵥβ₃, or the neurotensin receptor (NTR) would enhance the specificity and sensitivity for prostate cancer detection. This hypothesis was tested through the generation and characterization of superparamagnetic iron oxide nanoparticle (SPION) imaging agents. We successfully produced SPIONs which recognized PSMA and generated specific MRI contrast changes in images of cultured human prostate tumor cells, and in xenografts grown from these cells in nude mice. The bulk of the results have either been published (see progress report publications) or are in preparation so that only those studies which are directly related to this competitive renewal are summarized below. The specific Aims were:

Aim 1. To develop magnetic imaging Agents, consisting of super-paramagnetic iron oxide nanoparticles (SPIONs) conjugated with recognition ligands to prostate tumor epitopes (See above) and to select the best single Agent, or binary mixture of Agents, by measuring the relaxation times, contrast and magnetic fields generated by Agents binding to human prostate cancer cell lines in vitro.
Aim 2. To perform in-vivo MR and SQUID imaging of human prostate tumor xenografts in nude mice demonstrating that the binding of recognition-ligand conjugated SPIONs provides tumor specific contrast.
Aim 3. To quantitatively compare the performance of MR vs. SQUID imaging for tumor cell density measurement.

In support of Aim 2, we detected the binding of these PSMA-labeled SPIONs to LNCaP tumors in living mice by means of MR imaging, as shown in FIG. 3A, where the tumor appeared on the lower right of the Left image. After SPION injection into the tail vein, the tumor markedly brightened (FIG. 3A, Right). No contrast changes were seen in control mice bearing either PC3 or DU145 tumors, which do not express PSMA (Table 1).

TABLE 1
Cell Surface Epitope Densities

| Cell Line | PSMA | PSCA | αᵥβ₃ | NTR |
|---|---|---|---|---|
| LNCaP | 1.1E+06 | 0 | 1.1E+04 | 67.9 |
| DU-145 | 2.5E+04 | 0 | 1.2E+04 | 37.45 |
| PC3 | 1.4E+04 | 0 | 7.7E+03 | 15.85 |
| C4-2 | 1.7E+06 | 0 | 1.6E+04 | 79.26 |

**all numbers are the mean sites per cell for triplicate samples except NTR, which is mean channel fluorescence

The time course for the changes in the intensities of MR image signals for a similar mouse bearing dual LNCaP tumors (one on each flank) is shown in FIG. 4A. Both tumors brightened within the first imaging period, and increased in brightness after 24 hours. Note that there was no significant brightening of the control, muscle tissue during this time period. The intensities of both tumors returned to pre-injection values by 50 hours after injection, indicating that the SPIONs were cleared from the tumor after this length of time.

### Nanoparticle Synthesis: SIPPs

Although we successfully utilized commercial preparations of SPIONs to accomplish many of our early studies, we quickly realized that our lack of control over the magnetic, physical, and biochemical properties of these nanoparticles limited our ability to innovate. We therefore set out to prepare our own particles with properties tailored to our specific needs. Rather than duplicate preparations already in the literature, or commercially available, we sought to generate nanoparticles with unique, superior magnetic and biochemical properties, with the result that we chose to synthesize novel super-paramagnetic iron platinum particles (SIPPs). For MRI contrast agents, a higher magnetic moment at a given magnetic field causes larger perturbations in the magnetic relaxation times of nearby water protons and, thus, higher moment particles should generate increased image contrast. SIPPs have previously been reported with volume magnetizations greater than 590 emu/cm³, with some preparations approaching 1,140 emu/cm³, the saturation magnetization of bulk FePt (Xu et al. 2009; Zhao et al. 2009; Barmak et al. 2004; Zeng et al. 2002). These reported high magnetic moments suggested that SIPPs would be superior MRI contrast agents. We therefore synthesized a number of different SIPP preparations (Taylor et al. 2011; 2012) and measured their magnetic properties compared to a commercial preparation of MACS particles used earlier.

Magnetic Relaxivities were determined from the longitudinal and transverse relaxation times of μMACs SPIONs and our SIPPs at 4.7 Tesla. The measured SIPP relaxivities showed at least a 3-fold increase in r₂ and in the r₂/r₁ ratio (Table 2) suggesting that the SIPPs would be superior contrast agents for in vivo T₂-weighted imaging, compared with commercially available μMACs SPIONs.

TABLE 2
Particle Relaxivities at 4.7 Tesla

| Variable | Unit | μMAC | SIPP#2 |
|---|---|---|---|
| r₁ | Hz/mM | 1.67 | 1.18 |
| r₂ | Hz/mM | 21.37 | 62.2 |
| r₂* | Hz/mM | 436.09 | 253 |
| r₂/r₁ | Dimensionless | 12.81 | 52.58 |
| mass magnetization | emu/gram Fe | 81.7 | 69.2 |
| volume magnetization | emu/cm³ | 430 | 1038 |

### Encapsulation of SIPP Cores to Create Stealth Immunomicelles.

We synthesized SIPPs and encapsulated them with both polyethyleneglycolated, and rhodamine-conjugated, distearoyl-phosphatidyl-ethanolamine (DSPE) (FIG. 5A) to create stealth immunomicelles (DSPE-SIPPs) that could be specifically targeted to human prostate cancer cell lines and detected using both MRI and fluorescence imaging (Taylor et al. 2011; 2012). SIPP cores and DSPE-SIPPs were 8.5 nm±1.6 nm and 42.9 nm±8.2 nm in diameter and the SIPPs had a magnetic moment of 120 A-m²/kg iron. J591, a monoclonal antibody against prostate specific membrane antigen (PSMA), was conjugated to the DSPE-SIPPs (J591-DSPE-SIPPs) and specific targeting of J591-DSPE-SIPPs to PSMA-expressing human prostate cancer cell lines was demonstrated using fluorescence confocal microscopy (Taylor et al. 2011). The transverse relaxivity of the DSPE-SIPPs, measured at 4.7 Tesla, was 300.6±8.5 Hz mM⁻¹, which was 14-fold better than commercially available SPIONs (21.4±6.9 Hz mM⁻¹) and ˜3-fold better than reported relaxivities for Feridex® and Resovist®. Our data also show (FIG. 6A) that J591-DSPE-SIPPs specifically target human prostate cancer cells in vitro, are superior contrast agents in T₂-weighted MRI, and can be detected using fluorescence imaging. This is the first report on the synthesis of multifunctional SIPP micelles and using SIPPs for the specific detection of prostate cancer.

Since our SIPPs were constructed with a hydrophobic core, we used these particles to encapsulate drugs that were too hydrophobic to be used routinely for chemotherapy. One such class of drugs, the taxanes, have an attractive therapeutic profile, but are so insoluble in water that they must be administered in combination with adjuvants, such as cremophore. We have encapsulated a typical taxane, paclitaxel, into our particles, at a concentration that is equal to that used for chemotherapy, and prepared SIPPs targeted to PSMA. In order to monitor the specific uptake of our SIPPs, we made them with rhodamine-labeled phospholipids (red) and both fluorescent (green) paclitaxel and normal paclitaxel. These anti-PSMA conjugated SIPPs were specifically taken up by C4-2 cells (FIG. 6A; Top Row) where one notes both the red signal from the rhodamine lipids, and the green signal from the paclitaxel inside the cells. SIPPs conjugated with a non-targeting control antibody (IgG) were not taken up by the cells (FIG. 6A; Bottom Row).

These SIPPs also selectively altered the MRI contrast for PSMA-displaying xenografts in nude mice, in a manner similar to that shown above for SPIONs (FIGS. 3&4), specifically targeted these tumors (FIG. 7A) and selectively eradicated C4-2 tumors (FIG. 8A). The data in FIG. 8A show several important features of our work: (1) For the control particles containing either no drug, or no specific targeting agent, there is no effect on tumor growth. (2) Paclitaxel is effective against these xenografts, and (3) specific targeting of PSMA on these tumors with paclitaxel-containing particles eradicates these tumors in a PSMA-dependent manner.

We completed Aim 3 by measuring the MRI and SQUID responses to a number of nanoparticle-labeled human tumor cell lines in vitro. Our results indicated that MRI was at least 3-fold more sensitive than SQUID detection of the same tumor cells labeled with our nanoparticles. We also found that MRI detected all of the magnetic particles, while SQUID was able to only detect that fraction of iron oxide particles that had diameters of 24±2 nm. The SIPPs had diameters of 9 nm so that SQUID could detect less than 1% of these. For these reasons we are not proposing additional SQUID measurements.

### Preliminary Data for Therapeutics.

Recently, as part of our Natural Products-based Drug Development program at the University of New Mexico, we constructed libraries of analogs of the natural products curcumin and resveratrol as inhibitors of signaling pathways including NFκ-B (Weber et al., 2005,2006a,2006b; Heynekamp et al, 2006; Deck et al., 2008; Brown et al., 2008). Analogs from both libraries were identified as potent inhibitors of NF-κB; this involved determining their abilities to inhibit the TNFα-induced activation of NF-κB using the Panomics 293TNF-κB-luc screening cell. Analogs were identified that were up to 100-fold more potent NF-κB inhibitors than resveratrol or curcumin. One of the analogs from κ the curcumin library, designated ca27, was selected for study with prostate cancer cells (Fajardo et al., 2011). Some of the findings are:
1. Ca27 inhibited cell growth and induced cell death in the androgen dependent LNCaP cells (representative of early stage disease) and androgen ablation resistant C4-2 cells (representative of late stage disease) at concentrations of 5-10 μM (FIG. 1A; panels A and B).
2. At low miocromolar concentrations (<5 μM) ca27 rapidly down-regulated Androgen Receptor protein and prostate specific antigen (PSA) expression (FIG. 2A; panels A and B).

As described in the section on therapeutic applications of SIPPs, ca27 and other inhibitors of NF-κB will be incorporated in combination with taxanes into SIPPs for treatment of metastatic drug-resistant prostate cancer.

[chemical structure]

### Selection of Targeting Epitopes.

We have investigated several novel tumor cell surface epitopes [21] useful for targeting PCa in four cultured human prostate cancer cell lines: LNCaP, DU145, PC3, and C4-2. From the cell surface density (Table 1) and mRNA expression levels of PSMA, PSCA, the integrin αᵥβ₃, or NTR we concluded that PSMA was the most favorable, single target for prostate cancer detection. Therefore, our initial focus will be on the use of PSMA as the primary target for our nanotheranostics.

There are many reasons to choose PSMA as the first target. Prostate-specific membrane antigen (PSMA) is a prototypical cell-surface marker of prostate cancer. PSMA is an integral, non-shed, type 2 membrane protein with abundant and nearly universal expression in prostate carcinoma, but has very limited extra-prostatic expression. PSMA is widely recognized as an attractive molecular target for the imaging and treatment of metastatic prostate cancer [15] because: (1) it is abundantly expressed on more than 90% of prostate tumor cells. LNCap cells, for example express more than 1 million PSMA molecules per cell (Table 1). (2) Ligand-bound PSMA is recycled so that ligands bound to the extracellular portion trigger internalization of the PSMA molecule along with their bound cargoes. (3) This gives a convenient method for getting nanoparticle cargoes into prostate tumor cells. (4) PSMA's expression increases with the grade, stage and metastatsis of PCa so that more aggressive disease displays more targets for our nanotheranostics. (5) PSMA is not expressed to any large extant in non-cancerous tissues. (6) most solid tumors, even those of non-prostatic origin, display PSMA on their neovasculature, making PSMA a pan-tumor target. These attractive properties have spurred other attempts to develop of PSMA-targeted therapies for cancer, and first-generation products have entered clinical testing. Vaccine approaches have included recombinant protein, nucleic acid and cell-based strategies, and anti-PSMA immune responses have been demonstrated in the absence of significant toxicity. Therapy with drug-conjugated and radiolabeled antibodies has yielded objective clinical responses as measured by reductions in serum prostate-specific antigen and/or imageable tumor volume.

Furthermore, we have shown that green fluorescent FITC-labeled SPIONs targeted to PSMA using the humanized antibody J591 specifically bound to and were taken up by human prostate cancer cell lines [22]. AT 310 K, the particles were internalized with a half-time of 7 minutes (FIG. 1A) while at the lower temperature of 277 K, no uptake was seen. It was therefore with great interest that we next determined whether the recognition-ligand bearing SPIONs would actually seek out and specifically bind to tumors in vivo. We therefore grew LNCaP, C4-2, and DU145 human prostate tumor cell lines as xenografts in nude mice and injected anti-PSMA-conjugated SPIONs into the tail veins. LNCaP tumors took up the SPIONs, while the DU145 tumors, which lacked PSMA expression (Table 1) did not (FIG. 2A).

Neurotensin Receptor-1.

Radiotherapy combined with androgen depletion is generally successful for treating locally advanced prostate cancer. However, radioresistance that contributes to recurrence remains a major therapeutic problem in many patients. In this study, we define the high-affinity neurotensin receptor 1 (NTR1) as a tractable new molecular target to radiosensitize prostate cancers. The selective NTR1 antagonist SR48692 sensitized prostate cancer cells in a dose- and time-dependent manner, increasing apoptotic cell death and decreasing clonogenic survival. The observed cancer selectivity for combinations of SR48692 and radiation reflected differential expression of NTR1, which is highly expressed in prostate cancer cells but not in normal prostate epithelial cells. Radiosensitization was not affected by androgen dependence or androgen receptor expression status. NTR1 inhibition in cancer cell-attenuated epidermal growth factor receptor activation and downstream signaling, whether induced by neurotensin or ionizing radiation, establish a molecular mechanism for sensitization. Most notably, SR48692 efficiently radiosensitizedn PC-3M orthotopic human tumor xenografts in mice, and significantly reduced tumor burden. Taken together, our findings offer preclinical proof of concept for targeting the NTR1 receptor as a strategy to improve efficacy and outcomes of prostate cancer treatments using radiotherapy [20].

Imaging:

To develop SIPPs that are stable, sensitive, targeted MR imaging agents for all stages of prostate cancer. These SIPPs will be designed to:

1) Image tumor volume to reflect response to therapy; 2) Image tumor location(s) through delivery of targeted agents that alter MRI contrast; 3) Image drug delivery to a tumor and measure dose delivered, through Fe mapping; 4) Image residual disease, such as post-prostatectomy; and 5) Image tumor stage, extracapsular extension, seminal vesicles, lymph nodes and spinal or more distant metastases.

Synthesis of SIPPs:

We have previously described the development of SIPPs (Taylor et al.; 2011, 2012) and found that these superparamagnetic agents offer superior MRI contrast properties over superparamagnetic iron oxide nanoparticles (SPIONs). SIPPs will be synthesized using our previously published methods (Taylor et al. 2011; 2012). Briefly, 1.0 mmol Fe(NO₃)₃.9H₂O and 1.0 mmol Pt(Acac)₂ are added to 12.5 mmol ODA in a 25 mL 3-neck round bottom flask fitted with a reflux condenser. The reaction is heated to 330° C. (200° C./hr) with 10° C. recirculated cooling in the reflux condenser. Refluxing is continued for 45 minutes at which point the reaction is removed from the heat and allowed to cool to room temperature. The resulting black particles are collected in hexane and subjected to repeated washing by collecting particles in conical tubes with an external magnet, removing the supernatant, and resuspending in chloroform.

Encapsulation of SIPPs, Chemotherapeutics, and Experimental Drugs:

Phospholipid-encapsulated SIPP cores are prepared using a thin film method. 0.5 mL of SIPP cores (˜1% solids) in chloroform are added to a 20.0 mL glass scintillation vial. A chloroform mixture of 1,2-distearoyl-sn-glycero-3-phosphoethanolamine-N-[methoxy(polyethylene glycol)-2000] ((DSPE-PEG) for stealth capabilities), 1,2-distearoyl-sn-glycero-3-phosphoethanolamine-N-[biotinyl(polyethylene glycol)-2000] ((DSPE-PEG-biotin) for conjugating Antibodies/Peptide), 1,2-dipalmitoyl-sn-glycero-3-phosphoethanolamine-N-(lissamine rhodamine B sulfonyl) ((Liss-Rhod) for fluorescence), and 1-palmitoyl-2-(10,12-tricosadiynoyl)-sn-glycero-3-phosphoethanolamine (Diyne PE) for crosslinking using UV-light initiated photopolymerization) are then added to the hydrophobic cores. The mixture is further diluted in 0.5 mL of chloroform and vortexed thoroughly. For encapsulations with hydrophobic drugs, the procedure is the same except that 3.0 mg of the drug is also added to the mixture. Blowing a light nitrogen stream over the solution then evaporates the mixture. 5.0 mL of double-distilled water is heated to 80° C. and added to the thin film and vortexed thoroughly to hydrate the thin film. The resultant micelles are then extruded at 70° C. through an 80 nm nuclepore track-etch membrane filter using a mini-extruder to produce ˜45 nm micelles. The micelles are then purified from core-free micelles and excess phospholipids and drugs by collecting the magnetic particles using an LS magnetic column placed in a VarioMACS™ magnetic separator (Miltenyi Biotec, Carlsbad, Calif.). After the non-magnetic material has passed through the column, the particles are washed with water. The column is removed from the magnet and the purified SIPP-containing micelles are eluted in sterile saline. Variations in the development of encapsulated nanoparticles will include altering the nature of the phospholipids and incorporating neutral lipids, such as short-to-medium chain fatty acids or cholesterol, and/or crosslinkable phospholipids into the micelles.

Antibody Conjugation to Micelles:

Monoclonal antibodies or peptides against prostate cancer cell surface epitopes are conjugated to streptavidin in an overnight reaction using a Lightning-Link™ Streptavidin Conjugation Kit (Innova Biosciences, Cambridge, UK) according to the manufacturers' instructions. Concentrations of streptavidin, antibodies, and streptavidin-conjugated recognition ligands are quantitated using a NanoDrop™ 2000 Spectrophotometer (Wilmington, Del.). Streptavidin-conjugates are then incubated with the micelles overnight at 4° C. to conjugate the recognition ligands to the micelles through the biotin groups of the biotin-DSPE-PEG. A Micro BCA™ Protein Assay (Thermo Scientific, Rockford, Ill.) is used to quantitate the antibody concentrations and the amount of antibody conjugated to the micelle surface using a BioSpec-mini Spectrophotometer (Shimadzu, Columbia, Md.) at a wavelength of 562 nm.

Physical Characterization of SIPP Cores and DSPE-SIPPs:

Transmission electron microscopy (TEM) will be used to determine the size and polydispersity of the particle populations. For magnetic cores, a drop of the hexane suspension is applied to a carbon-coated grid and dried. For micelles, a drop of the aqueous suspension is applied to a carbon-coated grid, dried for 10 minutes, and the excess absorbed using a kimwipe. Adding a drop of 2% Uranyl Acetate solution followed by a 2-minute drying period negatively stains the grid. The excess is removed and the grid is allowed to dry for at least 5 minutes. The samples will then be imaged on a Hitachi 7500 transmission electron microscope with an acceleration voltage of 80 kV. Particle diameters will be calculated using ImageJ Software. At least 100 particles will be counted and the mean Feret diameters and standard deviations calculated. Diameters and zeta potentials of the micelles will additionally be measured using a Dynamic Light Scattering (DLS) instrument with zeta potential quantitation capabilities. The compositions of the SIPPs, phospholipids, drugs, and micelles will be investigated with thermogravimetric analysis (TGA) and differential scanning calorimetry (DSC). Aliquots of the micelles and their components (drug, phospholipids, magnetic cores) will be placed in TGA sample cups and evaporated at 30° C. under an argon stream for at least 90 minutes until all solvent has been removed and the mass of the sample stabilized. Weight loss profiles will then be measured under argon flow. The micelle content will be determined by measuring the mass loss profile while the temperature is raised from 30° C. to 1000° C. at a 10° C./min ramp rate. Inductively coupled plasma-optical emission spectroscopy (ICP) will be used to measure the metal content (iron and platinum) of each synthesis. Prior to analysis, aliquots of the particles are digested at 180° C. with nitric and hydrochloric acids in PDS-6 Pressure Digestion Systems (Loftfields Analytical Solutions, Neu Eichenberg, Germany). After cooling, the samples are made up to a known volume, mixed and centrifuged. Samples are then analyzed using a PerkinElmer Optima 5300DV ICP-OES using the recommended wavelengths for each of the analytes.

Magnetic Characterization of SIPP Cores and Micelles:

Superconducting quantum interference device (SQUID) magnetometry will be employed to measure the blocking temperatures and saturation magnetizations of the magnetic cores and micelles. An aliquot of the samples is applied to the end of a cotton Qtip® (Unilever, Englewood Cliffs, N.J.). Magnetic measurements will be made on a Quantum Design MPMS-7 SQUID magnetometer. Temperature sweeps between 0 and 310 K will be performed by zero-field cooling the sample and then measuring the magnetic moment as a function of temperature under the influence of a weak magnetic field (1 mT) during warming and subsequent cooling. This procedure will yield both a zero-field-cooled (ZFC) and field-cooled (FC) curve, respectively. Values of the blocking temperature (T_{B}) will be recorded by determining the peak location in each ZFC curve. Saturation magnetizations will be measured at 310 K (37° C.) by varying the applied field from −5 to 5 Tesla. Mass magnetizations will be calculated from the iron concentrations determined by ICP.

Magnetic Resonance Relaxometry:

Increasing concentrations of the different SIPP-, and drug-containing micelles will be added to 1% agarose in 2.0 mL self-standing micro-centrifuge tubes (Corning, Corning, N.Y.). Samples will be imaged on a 4.7 Tesla Bruker Biospin (Billerica, Mass.) MRI system with Paravision 5.1 software. Samples will be imaged with a 256×256 matrix, a variable TE, and TR=10 sec. T₁, T₂, and T₂* measurements will be acquired, respectively. The MRI samples will then be digested as above and the iron concentration determined with ICP. The relaxation rates, rₙ=1/Tₙ, will be calculated and plotted versus the ICP-determined iron concentration of each sample. Linear regression is used to fit the data and the relaxivity (rₙ) of each sample is given as the slope of the resulting line in units of Hz mM⁻¹ of iron.

Determination of Specific Binding to Human Prostate Cancer Cells:

Human prostate cancer cells will be seeded onto polylysine-coated cover slips in 6-well polystyrene plates (Corning, Corning, N.Y.) and incubated at 37° C., 5% CO₂ for 24 hours. The media will then be exchanged with media containing targeted and non-targeted SIPP- and drug-containing micelles. The cells will be incubated with the particles for 10 minutes at 37° C., 5% CO₂ and the media will then be aspirated unbound particles will be washed away from the cells. Cover slips will be mounted on slides containing a drop of ProLong® Gold Antifade Reagent with DAPI (Invitrogen, Eugene, Oreg.). Confocal Images will be acquired using a 60× oil objective with an Olympus IX-81 inverted spinning disk confocal microscope. Cells will also imaged by light microscopy, using a Zeiss Axiovert 25 CA inverted light microscope with a 63× phase-contrast objective.

Therapeutics: SIPPs that Deliver Therapeutics for Treatment of Drug Resistant Prostate Cancer In Vitro and In Vivo.

These SIPPs will be designed to:

1) Deliver drugs with poor bioavailability, such as paclitaxel, and improve their performance; 2) Deliver novel drugs that are toxic alone and improve their therapeutic potential through targeted delivery; 3) Deliver multiple drugs as a single cargo; and 4) Improve specificity by targeting more than one tumor-specific epitope.
Rationale for Combining a Taxane with Established Drugs and Experimental Agents:

The taxanes paclitaxel and docetaxel are used in the treatment of a wide range of cancers and are the last line of treatment for metastatic prostate cancer. Their cytotoxicity is attributed to cell cycle arrest through stabilization of microtubules; however, development of resistance limits the effectiveness of taxanes (Mancuso et al., 2007). Other mechanisms may also contribute to taxane cytotoxicity. Paclitaxel is known to induce soluble tumor necrosis factor alpha (sTNFα) production in macrophages. Building upon this observation, a recent study with breast cancer cell lines correlated activation of the TNFα pathway with taxane cytotoxicity but also with the development of resistance. Significantly, resistance to paclitaxel or docetaxel was linked to TNFα-induced activation of the pro-survival NF-κB signaling pathway. Moreover, inhibition of NF-κB reversed the resistance (Sprowl et al., 2012). Similar results were reported recently from studies of prostate cancer cell lines where the same conclusions were drawn, namely, that resistance to docetaxel is associated with activation of the NF-κB pathway (O'Neill et al., 2011). This suggests that treatment of metastatic taxane-resistant prostate cancer with a combination of a taxane with an inhibitor of NF-κB may be a promising therapeutic approach to metastatic prostate cancer that involves taxane-resistant tumors, and there is now substantial support for this idea (Caicedo-Granados et al., 2011; Fujiwara et al., 2011; Sreekanth et al., 2011).

The NF-κB family of transcription factors consists of homo- and hetero-dimeric combinations of five related proteins of the Rel family, p50, p52, p65/RelA, c-Rel, and RelB. The most prevalent activated form of NF-κB is the p50/p65 dimer, which is the product of the canonical or classical activation pathway. NF-κB is normally sequestered in the cytosol through complexation with inhibitors IκB, especially IκBα and IκBβ.

A variety of signals can modify IκB, resulting in liberation of NF-κB and its translocation to the nucleus where NF-κB regulates the expression of numerous pro-survival genes. Importantly, NF-κB is constitutively activated in numerous tumors. Many compounds are known to inhibit NF-κB signaling. The structures of these compounds are diverse, suggesting that they target different sites in this complex signaling pathway. In addition, there are cell-specific targets involved in regulation of the NF-κB pathway, suggesting the possibility to develop cell-specific inhibitors. Nevertheless, it is important to note that recent studies have shown, both for inflammation and cancer, that the roles of NF-κB, including specific roles for individual subunits of NF-κB, are complex and can be either pro- or anti-survival, depending on cell type, nature of the stress-inducing event, stage of the cancer and environment (Ben-Neriah and Karin, 2011; Perkins, 2012). This emphasizes that the therapeutic promise of targeting NF-κB, which has not yet resulted in any approved drugs, may be difficult to realize. This also emphasized that the targeted nanotechnological approach of this application may make an important contribution to addressing the complexity of targeting NF-κB.

We will use both clinically established chemotherapeutic drugs as well as experimental agents to test the ability of SIPP- and drug-containing micelles to enhance their efficacy through targeting and protection from clearance. Two established drugs used in the clinics are the two taxanes docetaxel and paclitaxel, as well as acylated derivatives such as DHA-paclitaxel and other derivatives (Bradley et al., 2001; Lim et al., 2009; Kuan et al., 2011), which convey significant survival benefits and improved response rates and quality of life (de Wit 2008). However, chemotherapy with these drugs tend to be not curative and to suffer from transient efficacy and substantial (cardio)toxicity. This ultimately is responsible for the reported annual mortality of prostate cancer patients of approximately 30,000 men and emphasizes the need for new therapeutic options. Although docetaxel and paclitaxel are clinically used, they also reflect agents that are quite hydrophobic which explains the many efforts to increase their bioavailability through nanomaterials (Gaucher et al. 2010). We thus expect our targeted approach to increase the in vitro and in vivo efficacy of these drugs. Most importantly, the long-term failure of taxanes for treatment of metastatic prostate cancer is the result of development of drug resistance, which now appears to be related to activation of NF-κB. Therefore, we propose to develop SIPP-micelles both for imaging and drug delivery. Specifically, the drug delivery protocol will be as follows: 1) begin with incorporation of taxanes into nanoparticles and demonstrate their improved effectiveness against metastatic prostate cancer that is still sensitive to taxanes; 2) repeat this study with metastatic prostate cancer that is taxane resistant, to gain a measure of the extent of resistance; and 3) repeat this study with metastatic prostate cancer that is taxane resistant but with use of nanoparticles that combine a taxane with an inhibitor of NF-κB.

[chemical structure]

Inhibitors of NF-κB and other active ingredients useful in the treatment of prostate cancer can be formulated as nanoparticles in accordance with the following three strategies.

Inhibitors of NF-κB Combined with a Taxane.

Examples of commercially available NF-κB inhibitors that are used in cell-based studies are BAY 11-7082 and SN-50. BAY 11-7082 is an inhibitor of cytokine-induced phosphorylation of IκB including TNFα. SN-50 is a peptide that contains the nuclear localization sequence (NLS) of p50 linked to the hydrophobic region (h-region) of the signal peptide of Kaposi fibroblast growth factor (K-FGF). The N-terminal K-FGF h-region confers cell-permeability, while the NLS (360-369) inhibits translocation of the NF-κB active complex into the nucleus. These are examples of the numerous commercially available inhibitors of NF-κB.

Repositioned Drugs, where the Proposed Target is NF-κB, Combined with a Taxane.

Drug repositioning, also called drug repurposing, is the examination of existing drugs for new uses. Drug repositioning is increasing as pharmaceutical companies see their drug pipelines drying up. Cardiac glycosides are a large family of naturally occurring compounds. Current use of cardiac glycosides is for treatment of patients with congestive heart failure and cardiac arrhythmias, where the mechanism appears primarily to involve inhibition of cardiac myocyte Na/K-ATPase, resulting in elevation of intracellular calcium (Riganti et al., 2011; Prissas et al., 2011; Mijatovic et al., 2006,2011; Zhi et al., 2010). There is emerging evidence that Na/K-ATPase has properties that are separate from its known catalytic function. Specifically, the alpha-subunit appears to be involved in multiple signaling pathways and is overexpressed in numerous cancers. A variety of targets have been suggested, including activation of NF-κB (Riganti et al., 2011). In support of this, UNBS 1450, a semi-synthetic cardenolide which is currently in clinical trials, has been shown to deactivate NF-κB in a number of cancer cells (Prissas et al., 2011). We recently reported that numerous cardiac glycosides are potent inhibitors of NF-κB, including oubain, digoxin and digitoxin (Shah et al., 2011). In addition, other compounds, such as indomethacin, have been shown to be promising repositioning drugs to combine with taxanes (Caicedo-Granados et al., 2011).

Inhibitors of NF-κB Combined with a Taxane.

Recently, as part of our Natural Products-based Drug Development program at the University of New Mexico, we constructed libraries of analogs of the natural products curcumin and resveratrol as inhibitors of signaling pathways including NF-B (Weber et al., 2005; 2006a; 2006b; Heynekamp et al, 2006; Deck et al., 2008; Brown et al., 2008). Analogs from both libraries were identified as potent inhibitors of NF-κB; this involved determining their abilities to inhibit the TNFα-induced activation of NF-κB using the Panomics 293T/NF-κB-luc screening cell. Analogs were identified that were up to 100-fold more potent NF-κB inhibitors than resveratrol or curcumin. One of the analogs from the curcumin library, designated ca27, was shown to effectively down-regulate the expression of the androgen receptor in prostate cancer cells (Fajardo et al., 2011); these results were described in the preliminary data section. This part of the proposed research will utilize various analogs of curcumin and resveratrol, such as ca27 and other analogs, all of which are potent inhibitors of NF-κB. The synthetic strategies have been reported. Ca27 is one of numerous inhibitors of NF-κB that we have developed, several of which are shown below.

[chemical structure]

[chemical structure]

Examples of Analogs of Resveratrol and Curcumin that are Potent Inhibitors of NF-κB.

### Development of Drug-Resistant Cell Lines.

The enhanced therapeutic efficacy of the targeted nanoparticles in this proposal will be tested against cells that are resistant to clinically used chemotherapeutic agents, such as docetaxel. We hypothesize that drug-loaded and targeted nanoparticles are able to inhibit prostate cancer cells and their tumors that are resistant to standard concentrations of chemotherapeutic agent. We will test this hypothesis by generating cell line subclones with enhanced resistance to standard concentrations of docetaxel. We will generate resistant cell lines as described previously (O'Neill et al. 2011). Briefly, the C4-2B and PC-3 cell lines will be cultured in standard medium (10% fetal bovine serum, antibiotics) supplemented with 4-8 nM docetaxel for 48 hours. The surviving cells are re-seeded. This treatment is repeated 5 times followed by another 5 treatment rounds at elevated docetaxel concentrations (8-12 nM). The resulting cells with enhanced resistance will be kept in the highest final concentrations used. Intermittent cell cultures will be set aside, maintained in liquid nitrogen, and utilized for comparative testing of resistance. The latter will be determined by standard cell proliferation, viability, and apoptosis assays, such as cell counting, DNA incorporation, trypan blue dye exclusion, nuclear histone leaking, etc. Alternatively, we have contacted Dr. O'Neill at the University College Dublin and she has agreed to share her newly established PC-3 and 22RV1 cell lines and make them available for our research (confirmatory letter available upon request).

### Drug Loading and Drug Release Rates:

Micelles will be dissociated by 3-fold dilution with acetonitrile, incubated at room temperature for 1 hr with occasional vortexing and centrifuged to collect the drug-containing supernatants. The solution will be filtered through a 0.2 μm syringe filter before HPLC quantification of drug content to measure the amount of drug loaded into the particles. For the drug release rate experiments, we will purify preparations of the drug/magnetic nanoparticle-containing micelles that have been incubated under different pH conditions with and without esterases on magnetic columns at 1, 3, 6, 12, 24, 48, and 72 hours post-synthesis. The particles will be eluted and resuspended in the original volume using mouse serum. The flow-through from the purifications will be analyzed using HPLC to determine the amount of drug released from the particles.

### Cytotoxicity:

Human prostate cancer cells will be cultured in 96-well plates. The following day, media will be exchanged with media containing the treatments or controls in one of five concentrations from 0.1 μM to 50.0 μM chemotherapeutic or experimental drug. We will determine, using HPLC and TGA (described above) the concentration of drug loaded into the particles and then use the same amount of iron and platinum (SIPPs) in the no-drug, control cytotoxicity assays. The cells will be collected 24 and 48 hours after the addition of the treatments or controls and an MTT assay will be used to quantitate the number of metabolically active cells in each sample and the concentration of Paclitaxel, or other drug, needed to inhibit the metabolic activity of 50% of the cells (IC₅₀).

In Vivo Dose Finding Experiments:

The highest effective dose (ED) of the chemotherapeutics and experimental drugs that can be injected into mice is determined. In Experiment A, we will use the IC₅₀ dose determined in vitro (1×) for injection into a single mouse. We will also use a 10× more concentrated dose (compared to the 1× dose) for injection into 1 additional mouse. We will also inject the dose into a 3^{rd} mouse that matches the amount of drug we are incorporating into the nanoparticles determined in our drug loading experiments. We will need 3 mice per drug or drug combination for Experiment A. These experiments will help us to find the highest non-lethal and generally non-toxic dose (ED). Next, using the highest non-lethal dose found in Experiment A, we will perform Experiment B for verification in 3 more mice (per drug combination) for a total of 6 mice per drug regime for the dose finding experiments, as summarized in Table 2 below.

TABLE 2

| | | | |
|---|---|---|---|
| Treatment | # mice | Dose | Description |

Experiment A (Per drug combination)

| | | | |
|---|---|---|---|
| Chemotherapeutic | 1 | 1X (IC₅₀ Dose) | The in vitro IC₅₀ Concentration |
| Chemotherapeutic | 1 | 10X | 10X more concentrated than the in vitro IC₅₀ |
| Chemotherapeutic | 1 | Drug Loading | The same concentration loaded into the micelles |

Experiment B

| | | | |
|---|---|---|---|
| Chemotherapeutic | 3 | ED | The highest non-lethal dose found in Experiment A |

Mice will be checked every other day for 20 days post injection (the time the actual study animals will be followed). If at any time the mice show signs of severe toxicity or distress the mice will immediately be euthanized and the adverse responses will be recorded for that specific dose.

### Biodistribution and Bioavailability:

Treatment and control groups of 3 mice each will be used for the initial biodistribution/bioavailability study. Athymic nude male mice will have clear, single-cell suspensions containing 3×10⁶ human prostate cancer cells in 50% Matrigel (Becton Dickinson, Bedford, Mass.) injected subcutaneously below one dorsal flank. Once tumors have reached a volume of 50 mm³, the mice will each be retro-orbitally injected with the ED found in the dose finding experiments. The mice will be sacrificed 24 hours post-injection and all organs and tumors will be collected and subjected to ICP and HPLC to quantify the amount of drug and SIPP cores in each tissue and tumor to measure the biodistribution and bioavailability.

### Xenograft Efficacy and MRI Contrast Enhancement:

Athymic nude male mice will have C4-2 xenografts implanted as described above in the biodistribution and bioavailability study. When tumors have reached 50 mm³, the mice will have the ED dose of either the treatment or controls injected retro-orbitally. The mice will be imaged pre-injection, 1 hour post-injection, and 24 hours post-injection using a 4.7 Tesla MRI scanner at the BRaIN imaging center to measure the contrast enhancement in the tumors. This experiment will use 6 mice in each treatment and control groups, determined at the completion of goals 1 and 2. Daily, from the time xenografts are initiated through the end of the experiments, a digital caliper will be used to measure the length, width, and height of the tumors growing on the flanks of mice. The volume of the tumors will then be calculated using the volume of an ellipse, V=πabc, where a, b, and c are half the length, width, or height respectively. The mice will be followed for 20 days post-injection, at which time they will again be imaged with MRI to monitor the therapeutic response. The mice will then be sacrificed and tumors and organs will be subjected to ICP and HPLC to quantify the amount of targeted treatment, SIPP cores, and biodistribution. Changes in tumor volume for each of the groups of mice will be plotted as a function of day's post-compound injection. Comparisons will be made between the drug regimes and targeted SIPP-drug-micelle's ability to decrease the volume of the tumors compared to controls.

### Orthotopic Efficacy and MRI Contrast Enhancement:

Athymic nude male mice will have 3×10⁶ human prostate cancer cells implanted by making a small incision in the skin followed by a small burr hole in the right femur and slowly injecting the cells. The hole will be filled with bone wax and the incision will be closed with sutures. 30 days post-implantation, the mice will have the ED dose of either the treatment or controls injected retro-orbitally. The mice will be imaged pre-injection, 1 hour post-injection, and 24 hours post-injection using a 4.7 Tesla MRI scanner at the BRaIN imaging center to measure the contrast enhancement in the tumors. The experiments will use 6 mice in each treatment and control group. The volume of the tumors will be measured using the MR images. The mice will be followed for 20 days post-injection, at which time they will undergo MR′ to, once again, measure the tumor volume. The mice will then be sacrificed and tumors and organs will be subjected to ICP and HPLC to quantify the amount of targeted treatment and SIPP or SPION cores. Changes in tumor volume for each of the groups of mice will be plotted as a function of day's post-compound injection. Comparisons will be made between the drug and targeted SIPP-drug-micelle's ability to decrease the volume of the tumors compared to controls.

### Summary:

We propose to extend our exciting early nanoparticle research, which produced multiple prostate cancer specific targeted superparamagnetic MR imaging agents, to studies of targeted nanoparticle agents that incorporate multiple functions, including MR imaging, fluorescence, and therapy of prostate tumors. We anticipate that targeted delivery of multifunctional superparamagnetic imaging agents containing paclitaxel, or other potentially-useful drugs, combined with an inhibitor of NF-κB will provide specific and sensitive prostate cancer detection and enhanced therapy, for both early, organ-confined, and disseminated, metastatic prostate cancer. The imaging goals will be pursued by developing targeted nanoparticle agents that alter MRI contrast to image tumor location(s) and volume(s) to reflect response to therapy, to image drug delivery to a tumor and measure dose delivered, through Fe mapping, and to image residual disease, tumor stage, extracapsular extension, seminal vesicles, lymph nodes and spinal or more distant metastases. In addition, we propose to develop targeted nanoparticle agents that enhance delivery of poorly-bioavailable drugs, such as paclitaxel, or novel drugs that are toxic alone, and to improve their performance, and/or to deliver multiple drugs as a single cargo, with improvements to specificity by targeting more than one tumor-specific epitope.

### REFERENCES FOR EXAMPLE 2 AND BACKGROUND OF THE INVENTION

[1] Jemal A, Simard E P, Dorell C, Noone A M, Markowitz L E, Kohler B, Eheman C, Saraiya M, Bandi P, Saslow D, Cronin K A, Watson M, Schiffman M, Henley S J, Schymura M J, Anderson R N, Yankey D, and Edwards B K. Annual report to the nation on the status of cancer, 1975-2009, featuring the burden and trends in HPV-associated cancers and HPV vaccination coverage levels. Journal of the National Cancer Institute 105(2013) No. 3 PMID: 23297039
[2] U.S. Cancer Statistics Working Group. United States Cancer Statistics: 1999-2009. Incidence and Mortality Web-based Report. Atlanta (Ga.): Department of Health and Human Services, Centers for Disease Control and Prevention, and National Cancer Institute; 2013. Available at: http://www.cdc.gov/uscs. No PMID
[3] Puech P, Potiron E, Lemaitre L, Leroy X, Haber G P, Crouzet S, Kamoi K, Villers A. Dynamic contrast-enhanced-magnetic resonance imaging evaluation of intraprostatic prostate cancer: correlation with radical prostatectomy specimens. Urology. 2009 74:1094-9. PMID: 19773038
[4] Hess K R, Varadhachary G R, Taylor S H, Wei W, Raber M N, Lenzi R, Abbruzzese J L. Metastatic patterns in adenocarcinoma. Cancer. 106 (2006) 1624-33.PMID: 16518827
[5] Bubendorf L, Schöpfer A, Wagner U, Sauter G, Moch H, Willi N, Gasser T C, Mihatsch M J. Metastatic patterns of prostate cancer: an autopsy study of 1,589 patients. Hum Pathol. 2000 May; 31(5):578-83. PMID: 10836297
[6] Nicolas Michoux, Paolo Simoni, Bertrand Tombal, Frank Peetersi, Jean-Pascal Machiels, and Frédéric Lecouvet. Evaluation of DCE-MRI postprocessing techniques to assess metastatic bone marrow in patients with prostate cancer. Clinical Imaging 36(2012)308-315. PMID: 22726969
[7] Taylor R M, Huber D L, Monson T C, Ali A M, Bisoffi M, Sillerud L O. Multifunctional iron platinum stealth immunomicelles: targeted detection of human prostate cancer cells using both fluorescence and magnetic resonance imaging. J Nanopart Res. 2011 Oct. 1; 13(10):4717-4729. PMID: 22121333
[8] Taylor R M, Huber D L, Monson T C, Esch V, Sillerud L O. Structural and Magnetic Characterization of Superparamagnetic Iron Platinum Nanoparticle Contrast Agents for Magnetic Resonance Imaging. J Vac Sci Technol B Nanotechnol Microelectron. 2012 March; 30(2):2C101-2C1016. PMID: 22872817
[9] Taylor R M, Sillerud L O. Paclitaxel-loaded iron platinum stealth immunomicelles are potent MRI imaging agents that prevent prostate cancer growth in a PSMA-dependent manner. Int J Nanomedicine. 2012; 7:4341-52. doi: 10.2147/IJN.S34381. Epub 2012 Aug. 6. PMID: 22915856
[10] Sprowl J A, Reed K, Armstrong S, Lanner C, Guo B, Kalatskaya I, Stein L, Hembruff S L, Parissenti A M (2012) Alterations in tumor necrosis factor signaling pathways are associated with cytotoxicity and resistance to taxanes: a study in isogenic resistant tumor cells. Breast Cancer Res 14:R2 PMID: 22225778
[11] Valkenburg K C, Steensma M R, Williams B O, Zhong Z. Skeletal Metastasis: Treatments, Mouse Models, and Wnt Signaling. Chin J Cancer. 2013 Jan. 18. doi: 10.5732/cjc.012.10218. PMID: 2332779
[12] Xu C, Yuan Z, Kohler N, Kim J, Chung M A, Sun S. (2009) FePt nanoparticles as an Fe reservoir for controlled Fe release and tumor inhibition. J Am Chem Soc 131 (42), 15346 PMID: 19795861
[13] Zhao F, Rutherford M, Grisham S Y, Peng X. (2009) Formation of monodisperse FePt alloy nanocrystals using air-stable precursors: fatty acids as alloying mediator and reductant for Fe3+precursors. J Am Chem Soc. April 15; 131(14):5350-8. PMID: 19301824
[14] K. Barrnak, J. Kim, L. H. Lewis, K. R. Coffey, M. F. Toney, A. J. Kellock, and J. U. Thiele, (2004) in Magnetism and Magnetic Materials Conference (Anaheim, Calif., USA) no PMID
[15] Zeng H, Li J, Liu J P, Wang Z L, Sun S. (2002) Exchange-coupled nanocomposite magnets by nanoparticle self-assembly. Nature 420 (6914), 395 PMID: 12459779
[16] Weber W W, Hunsaker L A, Abcouwer S F, Deck L M, Vander Jagt D L (2005) Anti-oxidant activities of curcumin and related enones. Bioorg Med Chem 13, 3811-3820. PMID:15863007
[17] Weber W W, Hunsaker L A, Roybal, C N, Bobrovnikova-Marjon E V, Abcouwer S F, Royer R E, Deck L M, Vander Jagt D L (2006) Activation of NFkB is inhibited by curcumin and related enones. Bioorg Med Chem 14, 2450-2461. PMID:16338138
[18] Weber W W, Hunsaker L A, Gonzales A M, Heynekamp J J, Orlando R A, Deck L M, Vander Jagt D L (2006) TPA-induced up-regulation of activator protein-1 can be inhibited or enhanced by analogs of the natural product curcumin. Biochem Pharmacol 72, 928-940. PMID:16934760
[19]. Heynekamp J J, Weber W W, Hunsaker L A, Gonzales A M, Orlando R A, Deck L M, Vander Jagt D L (2006) Substituted trans-stilbenes, including analogs of the natural product resveratrol, inhibit the human tumor necrosis factor alpha-induced activation of transcription factor nuclear factor kappB. J Med Chem 49, 7182-7189. PMID:17125270
[20] Deck L M, Hunsaker L A, Gonzales A M, Orlando R A, Vander Jagt D L (2008) Substituted trans-stilbenes can inhibit or enhance the TPA-induced up-regulation of activator protein-1. BMC Pharmacology 8, 19. PMID:19000313
[21] Fajardo A M, MacKenzie D A, Ji M, Deck L M, Vander Jagt D L, Thompson T A, Bisoff M. The curcumin analog ca27 down-regulates androgen receptor through an oxidative stress mediated mechanism in human prostate cancer cells. Prostate 2012 72:612-625. PMID: 21796654
[22] Mancuso A, Oudard S, Sternberg C N (2007) Effective chemotherapy for hormone-refractory prostate cancer (HRPC): present status and perspectives with taxane-based treatments. Crit Rev Oncol Hematol 61, 176-185. PMID: 17074501
[23] Wang Y, Wang X, Zhao H, Liang B, Du Q. Clusterin confers resistance to TNF-alpha-induced apoptosis in breast cancer cells through NF-kappaB activation and Bc1-2 overexpression. J Chemother. 2012 24(6):348-57. PMID: 23174100
[24] O'Neill A J, Prencipe M, Dowing C, Fan Y, Mulrane L et al (2011) Characterization and manipulation of docetaxel resistant prostate cancer cell lines. Mol Cancer 10, 126. PMID: 21982118
[25] Caicedo-Granados E E, Wuertz B R, Marker P H, Lee G S, Ondrey F G (2011) The effect of indomethacic on paclitaxel sensitivity and apoptosis in oral squamous carcinoma cells: the role of nuclear factor-kB. Arch Otolaryngol Head Neck Surg 137, 799-805. PMID: 21844414
[26] Fujiwara Y, Furukawa K, Shimada Y, lida T, Shiba H et al (2011) Combination paclitaxel and inhibitor of nuclear factor kB activation improves therapeutic outcome for model mice with peritoneal dissemination of pancreatic cancer. Pancreas 40, 600-607 PMID: 21343836
[27] Sreekanth C N, Bava S V, Sreekumar E, Anto R J (2011) Molecular evidences for the chemosensitizing efficacy of liposomal curcumin in paclitaxel chemotherapy in mouse models of cervical cancer. Oncogene 30, 3139-3152. PMID: 21317920
[28] Schorr K, Garcia-Pifieres A J, Siedle B, Merfort I, Da Costa F B. Guaianolides from Viguiera gardneri inhibit the transcription factor NF-kappaB. Phytochemistry. 2002 August; 60(7):733-40. PMID: 12127591
[29] Saadane A, Masters S, DiDonato J, Li J, Berger M. Parthenolide inhibits IkappaB kinase, NF-kappaB activation, and inflammatory response in cystic fibrosis cells and mice. Am J Respir Cell Mol Biol. 2007 June; 36(6):728-36. PMID: 17272824
[30] Bradley M O, Swindell C S, Anthony F H, Witman P A, Devanesan P,et al (2001) Tumor targeting by conjugation of DHA to paclitaxel. J Control Release 74, 233-23 6. PMID: 11489499
[31] Lim J, Chouai A, Lo S-T, Liu W, Sun X, Simanek E E (2009) Design, synthesis, characterization, and biological evaluation of triazine dendrimers bearing paclitaxel using ester and ester/disulfide linkages. Bioconjugate Chem 20, 2154-2161. PMID: 19877601
[32] Kuan C Y, Walker T H, Luo P G, Chen C F (2011) Long-chain polyunsaturated fatty acids promote paclitaxel cytotoxicity via inhibition of the MDR1 gene in the human colon cancer Caco-2 cell line. J Am Coll Nutr 30, 265-273. PMID: 21917707
[33] de Wit R. Chemotherapy in hormone-refractory prostate cancer. BJU Int. 2008 March; 101 Suppl 2:11-5. PMID: 18307687
[34] Gaucher G, Marchessault R H, Leroux J C. Polyester-based micelles and nanoparticles for the parenteral delivery of taxanes. J Control Release. 2010 April 2; 143(1):2-12. PMID: 19925835
[35] Riganti C, Campia I, Kopecka J, Gazzano E, Doudlier S, Aldieri E,Bosia A, Ghigo D (2011) Pleitropic effects of cardioactive glycosides. Curr Med Chem 18, 872-885. PMID: 21182478
[36] Prassas I, Diamandis (2008) Novel therapeutic applications of cardiac glycosides. Nature Rev Drug Disc 7, 926-935. PMID: 18948999
[38] Szczesna D, Ghosh D, Li Q, Gomes A V, Guzman G, Arana C, Zhi G, Stull J T, Potter J D. Familial hypertrophic cardiomyopathy mutations in the regulatory light chains of myosin affect their structure, Ca2+ binding, and phosphorylation. J Biol Chem. 2001 March 9; 276(10):7086-92. PMID: 11102452
[39] Shah V O, Ferguson J E, Hunsaker L A, Deck L M, Vander Jagt D L. Natural products inhibit LPS-induced activation of pro-inflammatory cytokines in peripheral blood mononuclear cells. Nat Prod Res. 2010, 24:1177-88. PMID: 20582811
[40] Shah V O, Ferguson, Hunsaker L A, Deck L M, Vander Jagt D L. Cardiac glycosides inhibit LPS-induced activation of pro-inflammatory cytokines in whole blood through an NF-κB-dependent mechanism. Int J Applied Res Nat Prod 2011, 4:11-19.
[41] Caicedo-Granados E E, Wuertz B R, Marker P H, Lee G S, Ondrey F G (2011) The effect of indomethacic on paclitaxel sensitivity and apoptosis in oral squamous carcinoma cells: the role of nuclear factor-kB. Arch Otolaryngol Head Neck Surg 137, 799-805. PMID: 21844414
[42] Shicheng Liu, Yiming Yuan, Yutaka Okumura, Norihiro Shinkai, and Hitoshi Yamauchi. Camptothecin disrupts androgen receptor signaling and suppresses prostate cancer cell growth. Biochemical and Biophysical Research Communications 394(2010)297-302. PMID: 20206136
[43] Lu G, Maresca K P, Hillier S M, Zimmerman C N, Eckelman W C, Joyal J L, Babich J W. Synthesis and SAR of (99m)Tc/Re-labeled small molecule prostate specific membrane antigen inhibitors with novel polar chelates. Bioorg Med Chem Lett. 2012 Sep. 13. pii: 50960-894X(12)01158-4. doi: 10.1016/j.bmc1.2012.09.014. PMID: 23333070
[44] Abdolahi M, Shahbazi-Gahrouei D, Laurent S, Sermeus C, Firozian F, Allen B J, Boutry S, Muller R N. Synthesis and in vitro evaluation of M R molecular imaging probes using J591 mAb-conjugated SPIONs for specific detection of prostate cancer. Contrast Media Mol Imaging. 2013 March; 8(2):175-84. doi: 10.1002/cmmi.1514. PMID: 23281290
[45] Kasten B B, Liu T, Nedrow-Byers J R, Benny P D, Berkman C E. Targeting prostate cancer cells with PSMA inhibitor-guided gold nanoparticles. Bioorg Med Chem Lett. 2013 Jan. 15; 23(2):565-8. doi: 10.1016/j.bmc1.2012.11.015. PMID: 23232055
[46] Wright G L Jr, Grob B M, Haley C, Grossman K, Newhall K, Petrylak D, Troyer J, Konchuba A, Schellhammer P F, Moriarty R. Upregulation of prostate-specific membrane antigen after androgen-deprivation therapy. Urology. 1996 August; 48(2):326-34. PMID: 8753752
[47] New ADC Effective against Prostate Cancer Cancer Discovery January 2013 3:OF11; 2012; doi:10.1158/2159-8290.C D-N B2012-137 no PMID
[48] Valerie N C, Casarez E V, Dasilva J O, Dunlap-Brown M E, Parsons S J, Amorino G P, Dziegielewski J. Inhibition of neurotensin receptor 1 selectively sensitizes prostate cancer to ionizing radiation. Cancer Res. 2011 Nov. 1; 71(21):6817-26. doi: 10.1158/0008-5472.CAN-11-1646. PMID: 21903767
[49] Serda R E, Adolphi N L, Bisoffi M, Sillerud L O. Targeting and cellular trafficking of magnetic nanoparticles for prostate cancer imaging. Mol Imaging. 2007 July-August; 6(4):277-88. PMID: 17711783
[50] Xu L, Josan J S, Vagner J, Caplan M R, Hruby V J, Mash E A, Lynch R M, Morse D L, Gillies R J. Heterobivalent ligands target cell-surface receptor combinations in vivo. Proc Natl Acad Sci USA 109(2012)21295-21300. PMID: 23236171
[51] Kurhanewicz, J., Swanson, M. G., Nelson, S. J., Vigneron, D. B. Combined magnetic resonance imaging and spectroscopic imaging approach to molecular imaging of prostate cancer. J. Magn. Reson. Imaging. 16: 451, 2002. PMID: 12353259
[52] Aydn H, Kzlgoz V, Tatar I G, Damar C, Ugan A R, Paker I, Hekimoglu B. (2012) Detection of prostate cancer with magnetic resonance imaging: optimization of T I-weighted, T2-weighted, dynamic-enhanced T1-weighted, diffusion-weighted imaging apparent diffusion coefficient mapping sequences and M R spectroscopy, correlated with biopsy and histopathological findings. J Comput Assist Tomogr. 2012 January-February; 36(1):30-45. PMID: 22261768
[53] Winter P M, Morawski A M, Caruthers S D, Fuhrhop R W, Zhang H, Williams T A, Allen J S, Lacy E K, Robertson J D, Lanza G M, Wickline S A. (2003a) Molecular imaging of angiogenesis in early-stage atherosclerosis with alpha(v)beta3-integrin-targeted nanoparticles. Circulation. November 4; 108(18):2270-4. PMID: 14557370
[54] Winter P M, Caruthers S D, Kassner A, Harris T D, Chinen L K, Allen J S, Lacy E K, Zhang H, Robertson J D, Wickline S A, Lanza G M. (2003b) Molecular imaging of angiogenesis in nascent Vx-2 rabbit tumors using a novel alpha(nu)beta3-targeted nanoparticle and 1.5 tesla magnetic resonance imaging. Cancer Res. September 15; 63(18):5838-43. PMID: 14522907
[55] Fajardo A M, MacKenzie D A, Ji M, Deck L M, Vander Jagt D L, Thompson T A, Bisoff M. The curcumin analog ca27 down-regulates androgen receptor through an oxidative stress mediated mechanism in human prostate cancer cells. Prostate 2012 72:612-625. PMID: 21796654
[56] Ozawa S, Imai Y, Suwa T, Kitajima M. What's new in imaging? New magnetic resonance imaging of esophageal cancer using an endoluminal surface coil and antibody-coated magnetite particles. Recent Results Cancer Res. 2000; 155:73-87. PMID: 10693240
[57] Morawski A M, Winter P M, Crowder K C, Caruthers S D, Fuhrhop R W, Scott M J, Robertson J D, Abendschein D R, Lanza G M, Wickline S A. Targeted nanoparticles for quantitative imaging of sparse molecular epitopes with MRI. Magn Reson Med. 2004 March; 51(3):480-6. PMID: 15004788
[58] Johansson L O, Bjornerud A, Ahlstrom H K, Ladd D L, Fujii D K. A targeted contrast agent for magnetic resonance imaging of thrombus: implications of spatial resolution. J Magn Reson Imaging. 2001 April; 13(4):615-8. PMID: 11276107
[59] Artemov D. Molecular magnetic resonance imaging with targeted contrast agents. (2003a) J Cell Biochem. 90, 518-24. PMID: 14523986
[59] Artemov D, Mori N, Ravi R, Bhujwalla Z M. (2003b) Magnetic resonance molecular imaging of the HER-2/neu receptor. Cancer Res. 63, 2723-7. PMID: 12782573
[60] Sillerud L O, McDowell A F, Adolphi N L, Serda R E, Adams D P, Vasile M J, Alam T M. 1H NMR Detection of superparamagnetic nanoparticles at 1 T using a microcoil and novel tuning circuit. J Magn Reson. 2006 August; 181(2):181-90. PMID: 16698297
[61] Serda R E, Bisoffi M, Thompson T A, Ji M, Omdahl J L, Sillerud L O. 1alpha,25-Dihydroxyvitamin D3 down-regulates expression of prostate specific membrane antigen in prostate cancer cells. Prostate. 2008 May 15; 68(7):773-83. doi: 10.1002/pros.20739. PMID: 18247401

### Example 3

### Theranostic Imaging of Metastatic Prostate Cancer

Theranostic imaging of metastatic prostate cancer (PCa) is conducted using a nanoplex platform that can ultimately be developed, modified, and applied for different cancers, different receptors, different pathways, and in combination with other treatments. Prostate specific membrane antigen (PSMA) is expressed on the membrane of androgen-independent metastatic PCa.

Our PSMA-targeted nanoplex carries a radiolabel for detection, siRNA to downregulate a specific pathway, and a prodrug enzyme that synthesizes a cytotoxic drug locally from a systemically administered nontoxic drug at the nanoplex site. Each component of the nanoplex is carefully selected to allow us to evaluate each of its aspects i.e. image-guided delivery of nanoplex, siRNA-mediated downregulation, and conversion of prodrug to cytotoxic drug by the prodrug enzyme, with noninvasive imaging. We selected the prodrug enzyme bacterial cytosine deaminase (bCD) since it converts a non-toxic prodrug 5-fluorocytosine (5-FC) to 5-fluorouracil (5-FU) that can be detected by 19F MRS. Because changes in choline metabolism can be easily detected clinically with magnetic resonance spectroscopic imaging (MRSI) and with [11C]choline PET imaging, and because choline kinase (Chk) is an important target in cancer, we have initially focused on using siRNA to downregulate choline kinase (Chk-siRNA).

### Methods:

Our prototype nanoplex is synthesized by conjugating three compartments: (i) the prodrug-activating enzyme bCD, (ii) the multimodal imaging reporter carrier poly-L-lysine (PLL) that carries [111In]DOTA for SPECT or [Gd3+]DOTA for MR and a fluorescent probe (Cy5.5 or rhodamine) and, (iii) the siRNA delivery vector: PEI (polyethyleneimine)-PEG (polythethyleneglycol) co-grafted-polymer [1]. These three compartments are covalently conjugated and siRNA-Chk is associated with the PEI-PEG co-grafted polymer through electrostatic affiliation. For PSMA-targeting, a low molecular weight urea-based PSMA targeting moiety (2-(3-[1-carboxy-5-[7-(2,5-dioxo-pyrrolidin-1-yloxycarbonyl)-heptanoylamino]-pentyl]-ureido)-pentanedioic acid (MW 572.56) [2] is used for conjugating NHS-PEGNHS (MW ˜3000) to PEI. Imaging studies with PSMA-targeted nanoplexes were performed with PC-3 human prostate cancer xenografts genetically engineered to overexpress PSMA (PC-3 Pip) in SCID mice. Non-PSMA-expressing PC-3 xenografts (PC-3 Flu) were used as controls. MR experiments were performed with a Bruker horizontal bore 9.4 T animal MR scanner using a home-built RF resonator. Fluorescence imaging was performed in vivo with a Xenogen IVIS Spectrum system. SPECT/CT images were acquired on a Gamma Medica X-SPECT scanner.

### Results and Discussion:

Images obtained with Pip and Flu tumors in FIG. 1XA demonstrate increased uptake in the PSMA-overexpressing Pip tumor compared to the non-PSMA-expressing Flu tumor. In separate studies we performed optical imaging of the nanoplex in tissue slices without or with PSMA blocking in mice with Pip and Flu tumors. Increased uptake in the Pip tumor compared to Flu was observed without blocking, which was reduced with blocking (FIG. 1XB). More specifically, FIG. 1XA illustrates SPECT imaging of SCID mouse bearing Pip (PSMA+ve) and Flu (PSMA-ve) tumor. Mouse was injected i.v. with 1.4 mCi of 111In labeled PSMA-targeted nanoplex (150 mg/kg in 0.2 ml). SPECT images were sec/projection. Following tomography, CT images were acquired in 512 projections to allow coregistration. Volume-rendered images were created using Amira image processing software. Decay-corrected volume-rendered SPECT/CT images at 48 h and 72 h demonstrate high liver uptake and specific accumulation in PSMA expressing Pip tumor.

FIG. 1XB shows nanoplex concentration in Pip and Flu tumors without (top panel) and with blocking (bottom panel). For the blocking studies 100 μg of anti-PSMAmouse monoclonal antibody (Clone GCP-05, Abcam) were injected i.v. in a PC3-Pip and PC3-Flu tumor bearing mouse. Five hours after injection, 1.5 mg of nanoplex (75 mg/kg) were injected i.v. in the same mouse. Mice were sacrificed 48 h after nanoplex injection. Tumors, muscle and kidney were excised and imaged on the Xenogen Spectrum system to detect rhodamine present in the nanoplex. Images are scaled differently for unblocked and blocked tissues. Corresponding quantitative information is shown in the bar graph in FIG. 1XB.

Administration of the theranostic nanoplex in mice bearing PC-3 Pip tumors resulted in a significant decrease of total choline (tCho) within 24 to 48 h, as shown in FIG. 2X. As shown in FIG. 2XA illustrates in vivo tCho maps from 2D CSI data sets acquired from a PC3-Pip tumor (˜400 mm3) before, 24 h, and 48 h after i.v. injection of the PSMA-targeted nanoplex (150 mg/kg) carrying bCD and Chk-siRNA. FIG. 2XB. shows tCho concentration calculated in arbitrary units before, 24 h, and 48 h after injection of nanoplex. Parameters used were echo time (TE)=120 ms, repetition time (TR)=1000 ms, 4 scans per phase encode step. CSI spectra were acquired at 9.4 T with an in-plane spatial resolution of 1 mm×1 mm from a 4 mm-thick slice.

The prodrug enzyme bCD converted the prodrug 5-FC to 5-FU at 24 h and at 48 h as shown in FIG. 3X. In vivo 19F MR spectra acquired from a PC3-Pip tumor (˜400 mm3) at (A) 24 h and (B) 48 h after i.v. injection of the PSMA-targeted nanoplex (150 mg/kg) carrying bCD and Chk-siRNA. Spectra were acquired after a combined i.v. and i.p. injection of 5-FC (450 mg/kg), on a Bruker Biospec 9.4 T spectrometer using a 1 cm solenoid coil tunable to 1H and 19F frequency. Following shimming on the water proton signal, serial nonselective 19F MR spectra were acquired with a repetition time of 0.8 s, number of scans, 2,000; spectral width, 10 KHz.

1. An immunocelle for treating prostate cancer comprising:
(a) a particulate core comprising a mixture of superparamagnetic particles and at least one active ingredient which is active for treating prostate cancer, said core being encapsulated by a plurality of phospholipds comprising at least one pegylated phospholipid, a phospholipid comprising conjugation functionalities and, optionally, a cross-linking agent; and
(b) a prostate cancer cell targeting monoclonal or polyclonal antibody which is conjugated to said particulate core through an appropriate functionality of the conjugatable phospholipid.

2. The immunomicelle of claim 1, wherein:
(a) the superparamagnetic particles are superparamagnetic iron platinum particles (SIPP), superparamagnetic iron oxide nanoparticles (SPIONs) or superparamagnetic manganese oxide particles (SMIONs);
(b) the active ingredient is active for treating metastatic prostate cancer; and
(c) the targeting antibody or peptide is a PSMA.

3. The immunomicelle of claim 1, wherein the active ingredient is a taxane which is optionally combined with an inhibitor of NF-κ pathway.

4. (canceled)

5. A method of treating prostate cancer comprising administering a composition according to claim 1 to a patient in need.

6. A method of simultaneously treating and imaging prostate cancer comprising co-administering to a subject in need thereof a pharmaceutical formulation comprising a plurality of the immunomicelles according to claim 1.

7. A method of diagnosing the presence and/or progression of anti-cancer treatment in a subject of prostate cancer comprising:
(a) administering a pharmaceutical formulation of the invention to the subject;
(b) subjecting the subject to magnetic resonance imaging; and
(c) determining through MRI contrast enhancement whether the subject suffers from prostate cancer and in particular, metastatic prostate cancer by comparing the resulting MRI image from the subject with a control or standard (which may be a disease control or a normal/healthy control to which the subject's MRI image may be compared for diagnosis).

8. A composition comprising a population of immunocells for treating prostate cancer, including metastatic prostate cancer, said immunoecelle comprising:
(a) a particulate core comprising an effective amount of an anticancer agent, optionally in combination with a NF-κB pathway inhibitor and further optionally in combination with a mixture of superparamagnetic particles, said core being encapsulated by a plurality of phospholipds comprising at least one pegylated phospholipid, a phospholipid comprising conjugation functionalities, and further optionally, a cross-linking agent, including a cross-linking phospholipid;
(b) a targeting antibody or peptide or other binding motif which is selected from the group consisting of prostate cancer cell targeting monoclonal or polyclonal antibody which is/are conjugated to said particulate core through an appropriate functionality of the conjugatable phospholipid.

9. The composition according to claim wherein 8 wherein said anticancer agent is a taxane which is combined with a NF-κB pathway inhibitor.

10. The composition according to claim 9 wherein said taxane is paclitaxel or docetaxel and said NF-κB pathway inhibitor is a compound selected from the group consisting of ca27, DHA-paclitaxel, BAY 11-7082, SN-50,

[chemical structure]

[chemical structure]

11. A method of determining the existence of cancer tissue in a patient comprising administering to said patient an effective amount of a population of paramagentic nanoparticles and subjecting said nanoparticles to NMR relaxometry to determine the volumetric quantitative MRI measurement of any superparamagnetic nanoparticle in biological tissues.

12. The method according to claim 11, wherein MRI measurements are taken of T₁-weighted (T_{1w}) and T₂-weighted (T_{2w}) images, the background relaxation times (T₁, T₂) of the tissue of interest and the relaxivity of the nanoparticles; the T_{1w}, and T₂, images are then converted into contrast images; and the contrast images are subtracted to yield the contrast difference.

13. The method according to claim 12, wherein calibration measurements of the effect of the selected superparamagnetic nanoparticles on water relaxation are used to determine the quantitative relationship between contrast difference and the concentration of the nanoparticles.

14. The method according to claim 13, wherein said quantitative relationship can be empirically inverted to yield the functional dependence of particle concentration on contrast difference, and said functional dependence is then used to convert the contrast difference image into an absolute nanoparticle concentration image.

15. The method according to claim 11, wherein said nanoparticles are SPIONS.

16. The method according to claim 11, wherein said cancer tissue is prostate cancer tissue or metastatic prostate cancer tissue.

17. (canceled)

18. A multifunctional superparamagnetic iron platinum nanoparticle (SIPP) comprising:
(a) two or more prostate cancer cell surface markers selected from the group consisting of prostate specific membrane antigen (PSMA), prostate stem cell antigen (PSCA), the integrin αᵥβ₃, and the neurotensin receptor (NTR); and
(b) one or more active ingredients selected from the group consisting of paclitaxel, docetaxel, ca27, DHA-paclitaxel, BAY 11-7082, SN-50, and one or more of the following compounds:

[chemical structure]

[chemical structure]

19. A stealth immunomicelle that specifically targets a human prostate cancer cell line and that is dectable by either MRI or fluorescence imaging, the immunomicelle comprising a multifunctional superparamagnetic iron platinum nanoparticle that:
(a) is encapsulated by polyethyleneglycolated and rhodamine-conjugated, distearoyl-phosphatidyl-ethanolamine (DSPE); and
(b) contains one or more prostate cancer cell surface markers selected from the group consisting of prostate specific membrane antigen (PSMA), prostate stem cell antigen (PSCA), the integrin αᵥβ₃, and the neurotensin receptor (NTR).

20. The stealth immunomicelle of claim 19, wherein the superparamagnetic iron platinum nanoparticle further comprises one or more active ingredients selected from the group consisting of paclitaxel, docetaxel, ca27, DHA-paclitaxel, BAY 11-7082, SN-50, and one or more of the following compounds:

[chemical structure]

[chemical structure]

21. (canceled)

22. (canceled)

23. A stealth immunomicelle that specifically targets a human prostate cancer cell line and that is dectable by either MRI or fluorescence imaging, the immunomicelle comprising a multifunctional superparamagnetic iron platinum nanoparticle that:
(a) is encapsulated by polyethyleneglycolated and rhodamine-conjugated, distearoyl-phosphatidyl-ethanolamine (DSPE); and
(b) contains one or more prostate cancer cell surface markers selected from the group consisting of prostate specific membrane antigen (PSMA), prostate stem cell antigen (PSCA), the integrin αᵥβ₃, and the neurotensin receptor (NTR) and J591;
wherein the superparamagnetic iron platinum nanoparticle has a core diameter of between about 5 nm to about 50 nm and the immunomicelle has a transverse relaxivity measured at 4.7 Tesla of between about 250 Hz mM⁻¹ to about 350 Hz mM⁻¹.

24. The stealth immunomicelle of claim 23, wherein the superparamagnetic iron platinum nanoparticle further comprises one or more active ingredients selected from the group consisting of paclitaxel, docetaxel, ca27, DHA-paclitaxel, BAY 11-7082, SN-50, and one or more of the following compounds:

[chemical structure]

[chemical structure]

25. The stealth immunomicelle of claim 24, wherein:
(a) the prostate cancer cell surface markers is prostate specific membrane antigen (PSMA) or J591; and
(b) the active ingredient is paclitaxel.

26. A pharmaceutical composition comprising a plurality of immunomicelles of claim 20 and, optionally, a pharmaceutically accecptable excipient.

27. A method of treating a subject who suffers from prostate cancer, the method comprising administering to the subject a pharmaceutically-effective amount of an immunomicelle of claim 20.

28. The method of claim 27, wherein the subject suffers from metastatic prostate cancer.

29. A method of diagnosing prostate cancer in a subject, the method comprising administering to the subject an amount of an amount of immunomicelles of claim 19 which is dectable by either MRI or fluorescence imaging.

30. A PSMA-targeted nanoplex comprising:
(a) a radiolabel for detection;
(b) a siRNA delivery vector comprising a siRNA which downregulates a specific pathway;
(c) a prodrug-activating enzyme that synthesizes a cytotoxic drug locally from a systemically administered nontoxic drug at a site targeted by the nanoplex; and
(d) a PSMA targeting moiety.

31. (canceled)

32. A method for theranostic imaging of metastatic prostate cancer (PCa) comprising administering to a subject who suffers from, or who is at risk of developing, metastatic prostate cancer a detactable amount of PSMA-targeted nanoplexes of claim 30.

33. A PEGylated stealth immunomicelle comprising:
(a) a particulate core comprising a mixture of superparamagnetic particles and at least one bioactive agent or drug comprising a lipid-modified drug selected from the group consisting of anti-cancer active agents and active agents useful in the treatment of prostate cancer, said core being encapsulated by a plurality of phospholipds comprising at least one pegylated phospholipid, a phospholipid comprising conjugation functionalities, and optionally, a fluorescence-inducing (fluorescent) phospholipid, and/or a cross-linking agent, including a cross-linking phospholipid; and
(b) a targeting antibody or peptide or other binding motif which is selected from the group consisting of a prostate cancer targeting monoclonal or polyclonal antibody and a monoclonal or polyclonal antibody or a peptide which targets prostate specific membrane antigen (PSMA), prostate stem cell antigen (PSCA), the integrin αᵥβ₃, and the neurotensin receptor (NTR) and which is/are conjugated to said particulate core through an appropriate functionality of the conjugatable phospholipid.

34. The immunomicelle of claim 33, wherein:
(a) the superparamagnetic particles are superparamagnetic iron platinum particles (SIPP), superparamagnetic iron oxide nanoparticles (SPIONs) or superparamagnetic manganese oxide particles (SMIONs);
(b) the active ingredient is selected from the group consisting of paclitaxel, docetaxel, ca27, DHA-paclitaxel, BAY 11-7082, SN-50, and one or more of the following compounds: [chemical structure] [chemical structure]
and
(c) the targeting antibody or peptide is a monoclonal or polyclonal antibody or a peptide which targets prostate specific membrane antigen (PSMA).

35. (canceled)

36. (canceled)

37. (canceled)

38. A pharmaceutical formulation comprising a plurality of the PEGylated stealth immunomicelles of claim 33 in combination with a pharmaceutically acceptable carrier, additive or excipient.

39. A pharmaceutical formulation comprising a plurality of PEGylated stealth immunomicelles of claim 35, in combination with a pharmaceutically acceptable carrier, additive or excipient.

40. The pharmaceutical formulation of claim 38, wherein the encapsulated particulate cores of each of the immunomicelles are cross-linked.

41. (canceled)

42. (canceled)

43. (canceled)

44. (canceled)

45. (canceled)

46. (canceled)

47. A method of diagnosing the presence or progression in a subject of a prostate cancer tumor comprising:
(a) administering a formulation of any of claim 39 to the subject;
(b) subjecting the subject to magnetic resonance imaging; and
(c) determining through MRI contrast enhancement whether the subject suffers from a prostate cancer tumor.

48. (canceled)

49. (canceled)

50. A method of diagnosing the presence or progression in a subject of a prostate cancer comprising:
(a) administering a formulation of claim 38 to the subject;
(b) subjecting the subject to magnetic resonance imaging; and
(c) determining through MRI contrast enhancement whether the subject suffers from a prostate cancer tumor.

51. (canceled)

52. (canceled)
